(19) United States
(12) Patent Application Publication
(10) Pub. No.: US 2008/0084539 A1
(43) Pub. Date: Apr. 10, 2008

(54) HUMAN-MACHINE INTERFACE DEVICE AND METHOD

(76) Inventor: Tyler Jon Daniel, Tokyo (JP)

Correspondence Address: TYLER DANIEL, DAITA 2-2-19, WHITE ROOM 105, SETAGAYA-KU, 155-0033, JP

(21) Appl. No.: 11/867,691

(22) Filed: Oct. 5, 2007

Related U.S. Application Data
(60) Provisional application No. 60/828,386, filed on Oct. 6, 2006.
(60) Provisional application No. 60/828,400, filed on Oct. 6, 2006.
(60) Provisional application No. 60/895,434, filed on Mar. 16, 2007.

(51) Int. Cl. A61B 3/14; G05B 15/00

(52) U.S. Cl. 351/210; 700/85

(57) ABSTRACT

The invention provides a coordinate input system, some embodiments having a first waveguide carrying stimulating light which is coupled by a force normal to the surface of the first waveguide into a second waveguide. In certain embodiments, the second waveguide contains photoluminescent material which upon receiving light from the first waveguide emits light which is detected by provided photosensors. Additionally, devices and methods for gaze tracking are provided having a probe element forming a probe image, an incident light sensing element for measuring the distribution of light incident at the location of the probe image, modulation and demodulation elements for distinguishing reflections of the probe image from extraneous light, and a comparison element for comparing the distribution of incident light to the probe image. The device is applicable to a gaze tracking apparatus which provides data useful in the field of user interfaces.

### CROSS-REFERENCE TO RELATED APPLICATIONS

The application claims the benefit of provisional patent applications Ser. Nos. 60/828,386, 60/828,400, 60/895,434, filed 2006 Oct. 6, 2006 Oct. 6, and 2007 Mar. 16, respectively, all by the present inventor.

### FEDERALLY SPONSORED RESEARCH

Not Applicable

### SEQUENCE LISTING OR PROGRAM

Not Applicable

### BACKGROUND OF THE INVENTION—FIELD OF INVENTION

The invention relates to the field of human-machine interface devices generally and more specifically to coordinate and gaze input devices.

### BACKGROUND OF THE INVENTION—PRIOR ART

Coordinate input devices are important in many fields, including computer user interfaces and mechanical systems. Many user interface devices for coordinate input exist, including the mouse, electronic tablet, light pens, and touch panels. Coordinate input devices are important in mechanical systems as evidenced by the widespread use of position encoders, angle encoders, velocity sensors and the like.

Existing coordinate input devices have numerous disadvantages including large volume, bulk, and high cost of manufacture.

One class of coordinate input device especially related to the present invention is the touch panel, particularly the transparent touch panel. Existing transparent touch panels can be grouped generally into four classes: capacitive, resistive, acoustic, and optical. Capacitive and resistive devices rely on transparent electrically conductive coatings of materials including ITO which are difficult and expensive to manufacture. Such systems also exhibit poor transparency. Acoustic systems show poor accuracy and are adversely affected by environmental factors including dirt and oil which can accumulate on the surface of the device. Existing optical systems are most often of the type forming a lamina of light above the interaction surface. These optical systems generally have poor accuracy and do not sense touch, but rather proximity resulting in poor usability. Another type of optical touch panel is based on frustrated total internal reflection (FTIR) using an out-of-plane imaging device and image processing algorithms to locate contact points. This type of device requires an expensive high-resolution camera, complex computer vision processing, and a large distance between the imaging sensor and interaction surface making it impractical for many applications. FTIR systems are described in U.S. Pat. Appl. 20030137494 by Tulbert, which is incorporated herein by reference.

Turning to gaze tracking, methods to accomplish various forms of gaze tracking have been known for over 20 years. The U.S. military has sponsored research with the goal of using gaze tracking in user interfaces, for example in aircraft cockpits. Advertisers have used gaze tracking systems to evaluate the effectiveness of advertising. The use of such systems for user interface design and evaluation is well known in the HCI (Human-Computer Interface) community. Perhaps the major application and driving force has been the use of gaze tracking technologies in user interfaces for the severely disabled, for example those persons unable to use their limbs.

Many different methods and devices to track the gaze of a viewer have been developed. One technology tracks the pupil of the eye and reflections or “glints” of one or more light sources on the cornea. When the gaze is directed towards a light source the corresponding glint will appear centered in the pupil. By measuring the distance and direction from the pupil center to the glint, the gaze direction relative to the light source can be determined. Most systems based on this technology use one or more cameras to measure the locations of the pupil and glint. However, to achieve acceptably precise results expensive, high-resolution cameras must be used. Processing the camera images also requires complex computer vision algorithms and computational hardware which increase cost, power consumption, and complexity. Some such systems use cameras of relatively low resolution but require that the camera be located very close to the viewer. This is restrictive and, when the camera is physically attached to the viewer, uncomfortable. Also, the use of such systems while wearing eyeglasses is often problematic, making the technique unsuitable for the large number people who wear corrective lenses or sunglasses. Related systems are described in U.S. Pat. No. 4,950,069 to Hutchinson and U.S. Pat. No. 5,220,361 to Lehmer, which are incorporated herein by reference.

Another gaze tracking technology tracks the shape of the iris as seen by a camera. When the eye gaze is directed towards the camera the iris appears circular; when the gaze is averted the iris appears elliptical. The shape of the pupil image can be used to determine gaze direction. This technology suffers from the same disadvantages as other camera-based techniques, for example high cost and system complexity.

Camera-based systems suffer from the additional disadvantage that cameras generally require lenses and other optical components to form an image at the imaging sensor surface. These components must be precisely mounted and are often responsible for a large part of the system's volume.

Still another eye movement sensing technology is electrooculography (EOG), which measures differences in electric potential that exist between the front and rear of the eye. This technology requires that sensors be mounted in close proximity to the eye, usually in contact with the skin, making the system intrusive and uncomfortable. Such a system in described in U.S. Pat. No. 5,726,916 to Smyth, which is incorporated herein by reference.

All of the above gaze-tracking technologies suffer from the additional limitation that they can measure only gaze direction and provide no information about the point or region upon which the eye is fixated. In other words, the above technologies do not distinguish between a blank stare in a certain direction and fixation on a point in the same direction.

In summary, there are currently no gaze tracking systems that are simple, non-intrusive, compact, and inexpensive to manufacture. Additionally, no systems known to the inventor are capable of detecting a point or region of fixation remotely.

### BACKGROUND OF THE INVENTION—OBJECTS AND ADVANTAGES

Accordingly, several objects and advantages of the present invention are:

(a) to provide a coordinate input method and device that can be implemented with good transparency to visible light;

(b) to provide a coordinate input method and device that is highly accurate;

(c) to provide a coordinate input method and device that can detect pressure;

(d) to provide a coordinate input method and device that can simultaneously detect multiple points of contact;

(e) to provide a gaze tracking method and device that does not require costly high-resolution imaging sensors;

(f) to provide a gaze tracking method and device that can be implemented compactly;

(g) to provide a gaze tracking method and device that works with a minimum of computational power, eliminating the need for expensive and power-hungry microprocessors;

(h) to provide a gaze tracking method and device that can track the point of fixation, not only the gaze direction; and

(i) to provide a gaze tracking method and device that operates remotely and does not restrict or cause discomfort to users.

### SUMMARY OF THE INVENTION

A new type of coordinate input device and method is described which may be simply and inexpensively implemented. The techniques described are scalable to large and small interaction surfaces, and the surfaces may have any form desired. The invention may be made transparent to a set of wavelengths of light, including transparency in the visible wavelengths. Several embodiments of the invention transmit light coupled into a waveguide at a point or points to photosensors and a signal processing unit which determines the location of the point or points.

Additionally a new type of gaze tracking device and method is described which has advantages in size, cost, complexity, and power consumption over existing devices and methods. A probe pattern is provided which, when observed by a viewer, is re-projected onto the original probe image. Features of the image are detected by low-power, compact optical sensors which determine the presence or absence of the re-projected image and, hence, the presence or absence of a viewer.

### BRIEF DESCRIPTION OF THE DRAWINGS

The invention will now be described, by way of example, with reference to the accompanying drawings, wherein:

FIG. 1A is an isometric view of a coordinate input device.

FIG. 1B is a schematic of a waveguide-photosensor response.

FIG. 1C is an empirically determined graph of normalized photosensor signal level vs. separation distance.

FIG. 1D illustrates the process of determining the unknown location of a contact point.

FIG. 1E shows a situation in which two photosensors are not sufficient to uniquely determine the location of a point.

FIG. 1F illustrates an appropriate photosensor coupling method.

FIG. 2 shows a side view of a waveguide into which light is coupled by scattering.

FIG. 3A shows an isometric view of a system using a patterned waveguide.

FIG. 3B illustrates of a waveguide pattern which produces a linear waveguide-photosensor response.

FIG. 4 illustrates the responses of two hypothetical photoluminescent materials to a pulse of incident stimulating light.

FIG. 5 illustrates waveguide stacking.

FIGS. 6A, 6B, and 6C show three signal layers.

FIGS. 7A, 7B, and 7C show the values and arrangement of two “button” signal layers.

FIGS. 8A, 8B, and 8C show a “button” embodiment where the button signal layers are not uniform.

FIG. 9A shows an embodiment using a light collecting element (LCE).

FIG. 9B illustrates the relationship of contact point location to the amount of light coupled into a LCE at each point along its length.

FIG. 9C shows an embodiment using multiple LCEs.

FIG. 9D shows two alternative methods of coupling LCEs to a sensing waveguide.

FIG. 11A shows an isometric view of a pressure-sensing embodiment.

FIG. 11B is a side view of a pressure-sensing embodiment.

FIG. 12 is a side view of an embodiment with a dual-function stimulating and sensing waveguide.

FIG. 13 shows a system using multiple light pressure distributions to increase the number of signal layers.

FIG. 14 shows an isometric view of a pressure-sensing system which uses a filter layer to provide multiple signal layers.

FIG. 15A is a top-view schematic of an embodiment using imaging sensors.

FIG. 15B illustrates the output of imaging system 1520.

FIG. 16A illustrates the various reflections of a contact point in a system capable of tracking at least two points simultaneously.

FIG. 16B shows the “worst case scenario” for the system in FIG. 16A.

FIG. 17 shows the various reflections of a contact point in a system capable of tracking at least four points simultaneously.

FIG. 18A illustrates an embodiment with a thin imaging system integrated with the sensing waveguide.

FIG. 18B shows an embodiment with an optical path folded under the transparent plane of the device leaving room for a display.

FIG. 19A is an isometric view of an embodiment that makes use of ambient light.

FIG. 19B is an isometric view of an alternative form of the embodiment of FIG. 19A.

FIG. 20 is a schematic of a camera-tracked interaction surface.

FIG. 21 is a schematic of a system to track remote objects.

FIG. 22 illustrates a method of display-touch panel calibration.

FIG. 23 is a diagram of the preferred embodiment in use by a viewer.

FIG. 24 illustrates an effect where the eye of a viewer forms an image of the scene being viewed both at the retina of the eye and at a focal plane containing the point of fixation.

FIG. 25 is a diagram illustrating how non-image-forming reflections evenly illuminate closely-spaced points distant from the surface of reflection.

FIG. 26 is a diagram of an alternative embodiment of the light sensing element of the present gaze tracking device.

FIG. 27A is a detailed view of the probe image of a preferred embodiment of the gaze tracking method described herein.

FIG. 27B shows the blurring effect of various processes occurring within a viewer's eye or optical imaging system.

FIG. 28A shows a view of the iris of a viewer's eye from the point of fixation.

FIG. 28B shows a view of the iris of the viewer's eye from FIG. 28A when the viewer's gaze has been averted.

FIG. 29 is a schematic of a prototype gaze tracking device.

### DETAILED DESCRIPTION

Definitions

As used herein, the following terms are intended to have the meanings as set forth below:

The term “light” is used in this document in its most general sense to mean “electromagnetic radiation.”

The term “gaze” refers to visual attention by a person, animal, or optical imaging system such as a camera.

The term “point of fixation” or “fixation point” refers to the point focused and centered in a field of view of a viewer.

Several mathematical conventions are used in this document. A single caret (ˆ) indicates exponentiation. integral_over_? indicates the integral over interval “?”. A variable quantity with a name of the form name_sub is equivalent to the variable name “name” with a subscript of “sub” in the attached figures. An asterisk indicates multiplication.

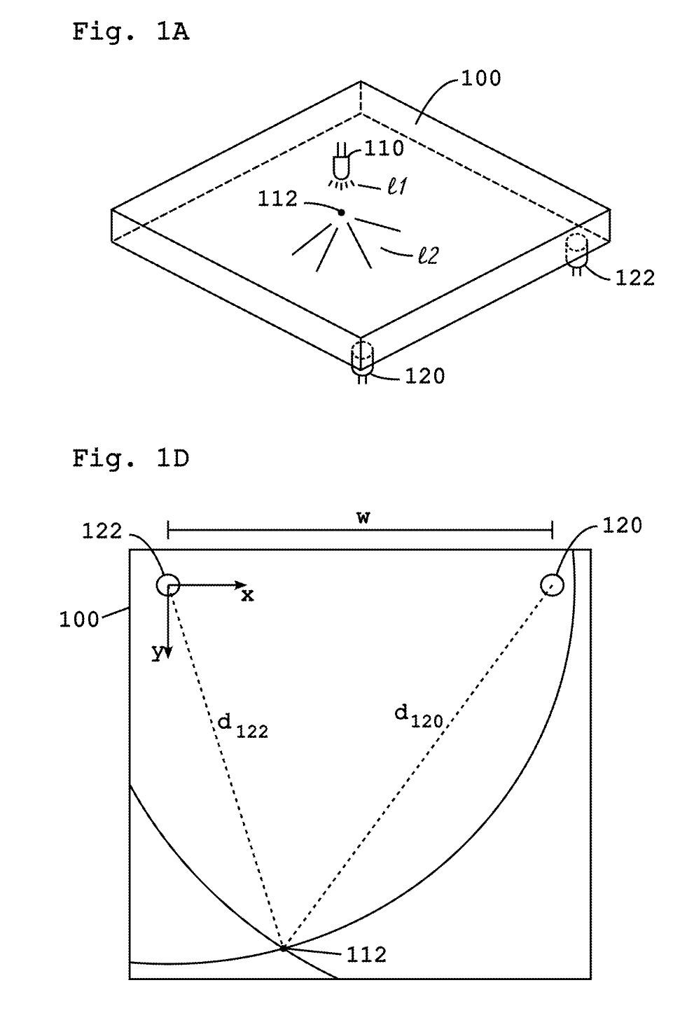

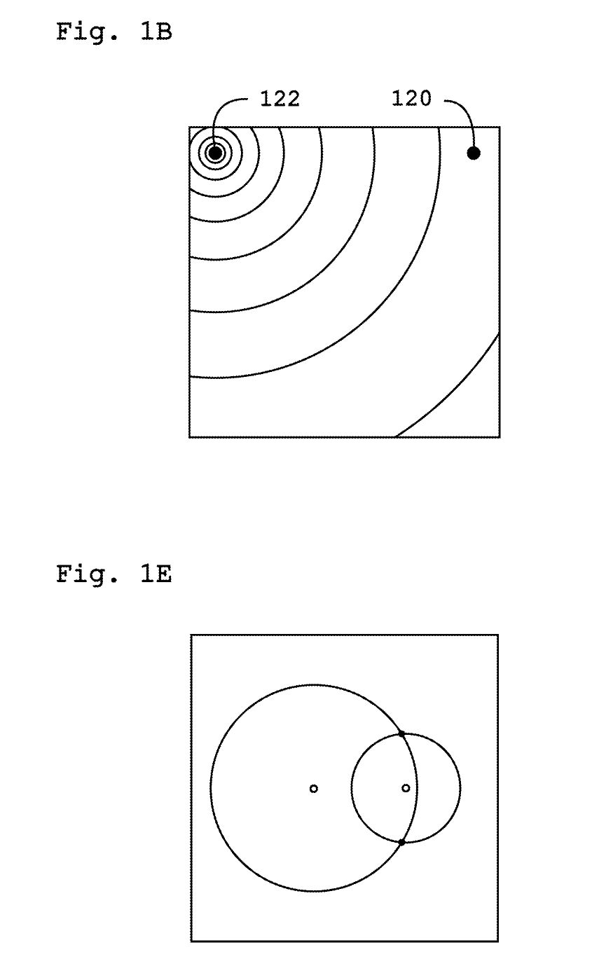

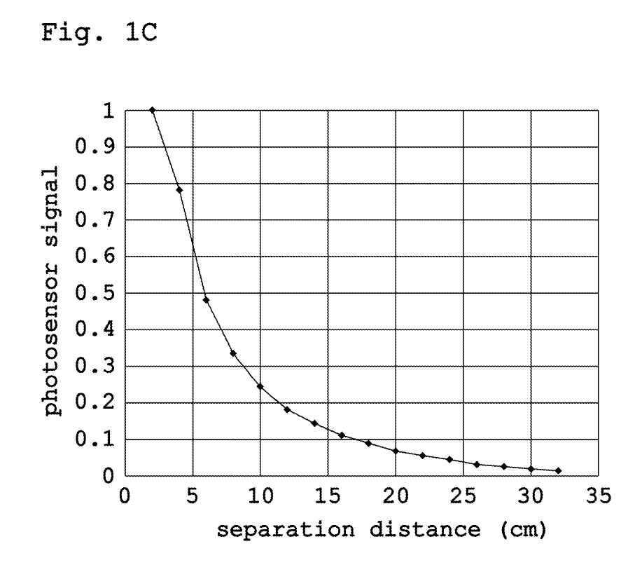

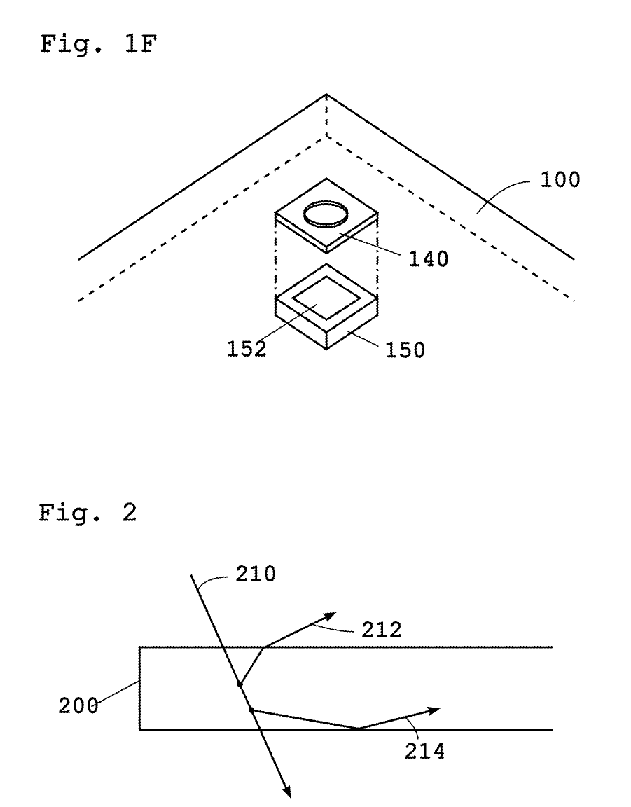

Turning to the drawings in detail in which like reference numerals indicate the same or similar elements in each of the several views, FIG. 1A depicts a position detector according to an embodiment of the present invention. A light source 110 emits light 11 which is incident on a waveguide 100 at point 112. Light 11 incident at point 112 with constant irradiance. Waveguide 100 contains photoluminescent material which partially or completely absorbs light 11 and isotropically emits light 12. Some of light 12 is trapped in waveguide 100 by total internal reflection (TIR) and propagates in all directions. Two photosensors 120 and 122 are configured such that portions of light 12 are absorbed by each photosensor and converted to electrical signals proportional to the amount of light reaching each photosensor. The edges of waveguide 100 are treated to suppress reflections such that the only portions of light 12 reaching photosensors 120 and 122 travel directly from point 112 to each photosensor. Because light 12 spreads out as it travels away from point 112, the amounts of light reaching photosensors 120 and 122 are inversely proportional to the distance between point 112 and the corresponding photosensor.

The distance from a photosensor to a point in the active area of an embodiment will hereafter be referred to as a separation distance. In the case of FIG. 1A, the precise relationship between separation distances and photosensor signals depends on many factors, including but not limited to: the composition and geometry of waveguide 100, the method of photosensor coupling, and the output of light source 110. Additional factors affecting the separation distance-photosensor signal relationship include the degree of scattering and absorption of light 12 as it travels through waveguide 100 and the number of surface irregularities such as scratches or dirt that cause light 12 to escape the waveguide. The separation distance-photosensor signal relationship may be determined by any of several methods including theoretical analysis, numerical simulation, and direct measurement.

The method of direct measurement may be used to empirically determine for each photosensor the relationship between all possible positions of point 112 and the resulting photosensor signals and is described hereafter. The photosensor signal is recorded as light source 110, and hence point 112, is swept across the surface of waveguide 100. The resulting data forms a map for each photosensor relating photosensor signal to the position of point 112 on waveguide 100. This map will be referred to as the waveguide-photosensor response. The waveguide-photosensor response may be expressed mathematically as a function of two variables and will henceforth be written as WP(x, y), where x and y are coordinates in a Cartesian plane containing waveguide 100. The mathematical representation may be a simple bilinear interpolation of empirically-determined data points, a polynomial function approximating the data points, or any other suitable representation.

FIG. 1B illustrates a typical waveguide-photosensor response. Each circular arc represents a set of locations of point 112 that produce identical signals in photosensor 122. The density of circular arcs represents the signal level produced by photosensor 122. In the case of FIG. 1B, the waveguide-photosensor response may be more simply expressed mathematically as WP(d), where d is the separation distance and d=sqrt(xˆ2+yˆ2). It is apparent from FIG. 1B that the signal from photosensor 122 increases as the separation distance decreases.

FIG. 1C is a graph of normalized photosensor signal level vs. separation distance empirically determined by the method of direct measurement described above for a prototype system configured as illustrated in FIG. 1A. The waveguide was constructed from a square sheet of Kuraray COMOGLAS 155K photoluminescent polymer 2 mm in thickness and 30 cm in dimension. Commonly available silicon photodiodes and an ultraviolet (395 nm) light emitting diode were used for photosensors 120 and 122 and light source 110, respectively.

The procedure for computing the unknown coordinates of point 112 from measured photosensor signals will now be described. First the waveguide-photosensor response is used to determine the distance from the associated photosensor to point 112. Mathematically, s_{—122=WP}_122(d_122), where s_122 is the measured signal of photosensor 122, WP_122 is the waveguide-photosensor response associated with photosensor 122 and waveguide 100, and d_122 is the separation distance from photosensor 122 to point 112. Hence, d_122=iWP_122(s_122), where iWP_122(d) is the inverse of WP_122(d). This may be understood graphically by noting that the known photosensor signal can be used in conjunction with FIG. 1C to look-up the separation distance d_122. This procedure is repeated for photosensor 120 to determine separation distance d_120.

Referring now to FIG. 1D, note that the separation distance determines for each photosensor a circular arc of possible locations for point 112. Point 112 is then located at the intersection of the two arcs centered at the locations of photosensors 120 and 122. This relationship may be expressed mathematically using the coordinate system illustrated in FIG. 1D with its origin at photosensor 122:
xˆ2+yˆ2=d_{—}122ˆ2
(x−w)ˆ2+yˆ2=d_{—}120ˆ2
where x and y are the coordinates of point 112 and w is the distance between photosensors 120 and 122. The active area is defined to be the set of points where 0<=x, y<=w. This system of equations is solved for the unknowns x and y using simple algebra, discarding solutions outside the active area. Note that in this case the two photosensors 120 and 122 suffice to uniquely determine the location of point 112. If the photosensors were positioned differently, however, as in FIG. 1E, two photosensors are in general not sufficient to uniquely determine the location of point 112 in a full plane containing the photosensors. Unique determination of point 112 in an arbitrary plane requires at least three photosensors, which is the more general, well-known case of trilateration. The calculation for determining the location of an unknown point in a plane using three photosensors simply adds to the system of equations above a third equation describing the additional separation distance. Solving the system of three equations uniquely determines the location of the unknown point. Note that there is no restriction on the shape of the active area or waveguide as stated in related art, and that the waveguide need not be planar.

Photosensors 120 and 122 are coupled to waveguide 100 in a manner such that the photosensor signals do not depend on the direction of point 112 with respect to the photosensor, but only on the separation distance. One appropriate coupling method is shown in FIG. 1F. A photosensor 150 with square sensing region 152 is attached to a circular aperture 140 which is in turn attached to the underside of a waveguide 100. The circular region in aperture 140 is composed of a clear material of refractive index high enough to allow light propagated by TIR in waveguide 100 to escape and pass through aperture 140, striking sensing region 152. Aperture 140 is attached directly to the core material forming waveguide 100 and not any cladding materials which may be present. Many commonly available photosensors have planar sensing surfaces and produce signals that vary strongly with the angle formed between incident light and the axis normal to the plane of the sensing surface. In the configuration illustrated in Fig IF the angular distribution of light striking photosensor 150 is constant with respect to the axis normal to the plane of sensing region 152.

Waveguide 100 has a refractive index greater than the surrounding medium and preferably greater than 1.3. One material suitable for the construction of waveguide 100 is polymethyl methacrylate (PMMA) dyed with DFSB-CO Clear Blue Fluorescent Dye available from Risk Reactor in Huntington Beach, Calif. Another suitable material for waveguide 100 is the aforementioned Comoglas 155K. Further information on suitable dyes and materials can be found in U.S. Pat. Appl. 20050123243 by Steckl et al., which is incorporated herein by reference. Photosensors 120 and 122 are preferably photodiodes sensitive to at least part of the spectrum of light l2, such as BPW-34 available from Siemens. Waveguide 100 may be clad with materials of lower refractive index to protect waveguide 100 from damage and/or contamination.

Waveguide 100 may be transparent to visible wavelengths of light, using materials such as those detailed above, partially opaque to visible light, or completely opaque to visible light, so long as it transmits some part of light l1 and light l2.

A further embodiment of the present invention replaces photoluminescent waveguide 100 described above with a waveguide that is not photoluminescent but partially scatters incident light. Note that the photoluminescent material in the previous embodiment served to couple light incident at a point on the surface into waveguide 100 by absorbing and isotropically re-emitting the incident light such that part of the re-emitted light was trapped by TIR. The present embodiment achieves this coupling through the phenomenon of scattering instead of photoluminescence. FIG. 2 is a side view of a waveguide 200 constructed according to the present embodiment. Light 210 is incident on waveguide 200. Part of light 210 passes through waveguide 200, while part is scattered. Light 212 is scattered but escapes waveguide 200, while other light 214 is scattered and trapped by TIR. The waveguide may be composed of a single material that scatters a significant part of incident light or a largely non-scattering, transparent material with embedded particles of opaque or scattering material. One example of a material with suitable intrinsic scattering properties is polyethylene (PE). An example of a suitable composite waveguide material is PMMA with small particles of embedded titanium dioxide (TiO2). The size of embedded particles may be adjusted to scatter only certain wavelengths of incident light. For example, embedded particles of TiO2 of sizes on the order of the wavelengths of near ultra-violet light (NUV) will tend to scatter NUV more than visible light, producing a waveguide transparent to visible light that can be used with a NUV light source. A further method of coupling light is roughening one or both surfaces of an otherwise largely non-scattering waveguide. Note that the techniques of the present embodiment scatter light incident at the waveguide surface and also light propagating along the waveguide, unlike the photoluminescent waveguide of the previous embodiment. Although the waveguide transmission characteristics are different, the techniques of the previous embodiment may still be applied to determine the waveguide-photosensor response and compute the coordinates of light incident at a point from the measured signals of photosensors coupled to the waveguide, also as in the previous embodiment. Hereafter, embodiments will be described using photoluminescent waveguides, however it is understood that the techniques of the present embodiment for coupling light into a waveguide may be applied where appropriate.

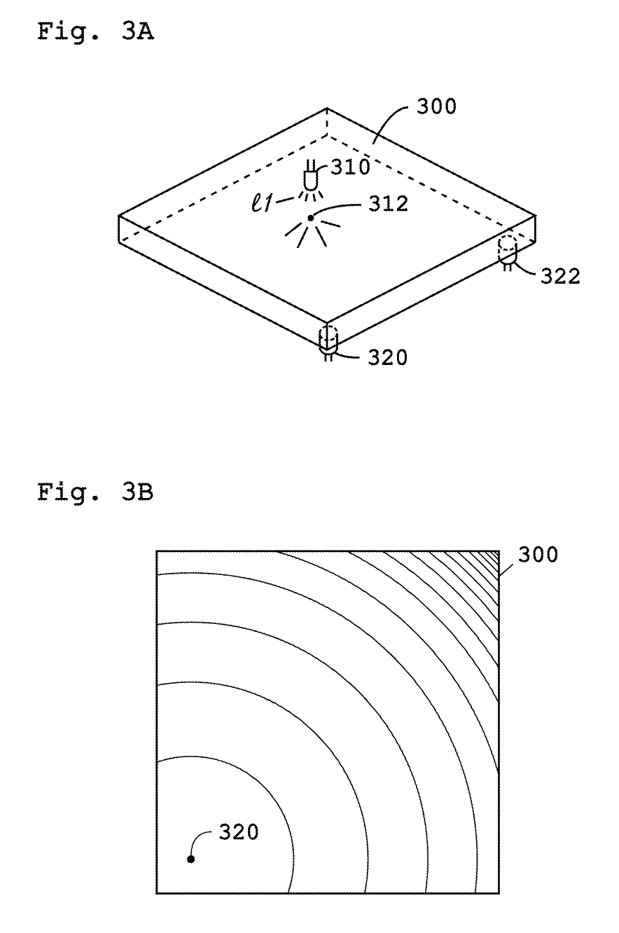

FIG. 3A is an isometric view of a further embodiment. As in the system depicted in FIG. 1A, a light source 310 emits light l1 incident at a point 312 on the surface of a photoluminescent waveguide 300. Two photosensors 320 and 322 are configured to receive light emitted by photoluminescence at point 312 and produce electrical output signals to be measured. Unlike the system of FIG. 1A, however, waveguide 300 has been non uniformly dyed or doped with two types of photoluminescent material A and B, each applied in a unique spatial distribution. Material A absorbs part of light l1 and re-emits light in a spectrum A. Likewise, material B absorbs part of light l1 and re-emits light in a spectrum B different from spectrum A. Put another way, materials A and B may have identical excitation spectra but must have unique emission spectra. Photosensor 320 is sensitive only to parts of spectrum A not present in spectrum B, and photosensor 322 is sensitive only to parts of spectrum B not present in spectrum A. Therefore, the output signal of photosensor 320 is proportional to the amount of light emitted by material A and not material B and, conversely, the output signal of photosensor 322 is proportional only to the amount of light emitted by material B. The distributions of materials A and B are controlled independently. Consequently, the waveguide-photosensor responses WP_320 and WP_322 may also be controlled independently, whereas in previous embodiments with a single photoluminescent material all waveguide-photosensor responses were linked.

Each waveguide-photosensor response in a system will henceforth be referred to as a “signal layer” and a waveguide with multiple signal layers will be said to be “multiplexed.” A method used to create independent signal layers will henceforth be referred to as a “method of signal separation” or a “signal separation method.” The signal separation method of the present embodiment separates the signal layers based on the unique emission spectrum of each signal layer and so will be referred to as “emission spectrum signal separation.” Note that waveguide 100 in FIG. 1A contains two signal layers and is therefore multiplexed; however, both signal layers of waveguide 100 are linked and not independently controllable by the system designer.

The density of material A at a given point determines the amount of light incident at that point which will be coupled into waveguide 300 and eventually converted by photosensor 320 into an output signal. Hence, the density of material A at a point adjusts or modulates the output signal of photosensor 320 due to light incident at the point. The process of non-uniformly adjusting the amount of incident light coupled into a waveguide will be referred to as “patterning” a waveguide or “waveguide patterning.”

The usefulness of waveguide patterning is illustrated in FIG. 3B, which shows a top view of waveguide 300 and photosensor 320, with contour lines roughly indicating the density of material A. Material A has been distributed in such a way that the waveguide-photosensor response of waveguide 300 and photosensor 320, WP_320, is a linear function of separation distance instead of the non-linear case of the first embodiment (FIG. 1C). The linear nature of WP_320 is advantageous in many systems, for example those involving analog-to-digital converters (ADCs) where linear output signals make best use of ADC resolution. Material B is similarly distributed such that WP_322 becomes a linear function of separation distance. The unknown location of point 312 is determined from the measured output signals of photosensors 320 and 322 using the same methods presented above for the first embodiment, the only difference being that the waveguide-photosensor responses are simpler, linear functions of separation distance.

A further embodiment of the present invention is similar to the previous embodiment illustrated in FIG. 3A in that two materials A and B are non-uniformly distributed over the surface of a waveguide. Unlike the previous embodiment, however, materials A and B may or may not have unique emission spectra, while they must have unique excitation spectra. In this case light is coupled into the waveguide by irradiating the waveguide with two different spectra of light, one spectrum inducing photoluminescent emission in material A and the other spectrum inducing photoluminescent emission in material B. The intensities of the two different spectra of light are independently modulated such that the output signals of photosensors coupled to the waveguide may be separated using demodulation techniques. One useful modulation technique involves alternately irradiating the waveguide with each spectrum of light. The photosensor output signals will therefore alternately correspond to the amount of light coupled into the waveguide by materials A and B and may be independently measured. Well-known modulation/demodulation techniques from the field of communications may also be employed, such as frequency or amplitude modulation of a carrier signal. This method of signal separation will be referred to as “excitation spectrum signal separation.”

A further embodiment of the present invention is a waveguide patterning technique for systems that couple incident light into a waveguide by scattering. In this embodiment the degree of scattering is varied over the surface of the waveguide to pattern the waveguide in a manner analogous to the varied distribution of photoluminescent materials described above. Methods of varying the degree of scattering in a waveguide include varying surface treatments such as roughening over the surface and constructing the waveguide of high- and low-scattering materials in varying ratios.

Still another waveguide patterning method is the selective blocking of light from a light source irradiating a waveguide. Methods of blocking incident light include interposing a filter layer of varying transparency between the light source and waveguide. The degree of transparency may be varied depending on the wavelength of light. One embodiment might, for example, consist of a transparent polymer dyed with three dyes, each blocking, respectively, red, green, or blue parts of the visible spectrum. The amount of light passed by the filter in each of these regions of the visible spectrum is controlled by varying the amounts of dye at each point in the polymer.

An additional embodiment of the present invention achieves signal separation by patterning a waveguide with photoluminescent materials of differing rise and/or decay times. The rise time of a photoluminescent material is the time between absorption of stimulating radiation and the emission of light at some fraction of its maximum level. The decay time is the amount of time elapsed after the stimulating radiation is removed until the amount of emitted light falls to some fraction of its maximum level. FIG. 4 illustrates the responses of two hypothetical photoluminescent materials to a pulse of incident stimulating light. The differing rise and/or decay times may be used to separate the signals from each material even when the materials have very similar emission and/or excitation spectra. One separation method records output signal levels from photosensors in the system at times t_1 and t_5. At time t_1, as illustrated in FIG. 4, material A is emitting light but not material B. At time t_5, material B is still emitting light while material A has completely decayed. This and other methods of separating the responses of different photoluminescent materials are well-known in the fluorescence microscopy community and described in Lakowicz, “Principles of Fluorescence Spectroscopy”, 2nd Ed., 1999, published by Springer which is incorporated herein by reference.

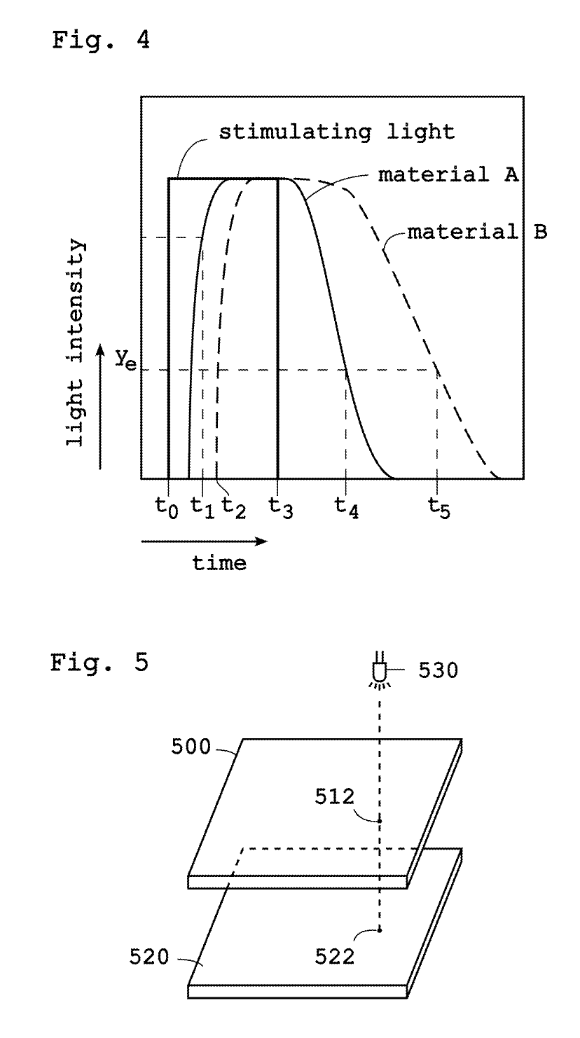

FIG. 5 shows an exploded isometric diagram of still another embodiment. Two waveguides 500 and 520 are positioned parallel and proximal to one another. A stimulating light source 530 emits light incident at point 512 where it is partially absorbed. Light from light source 530 not absorbed at point 512 travels through waveguide 500 to strike waveguide 520 at point 522, where it is at least partially absorbed. Light absorbed at points 512 and 522 is coupled into waveguides 500 and 520 by the mechanisms described above and measured using photosensors not shown in FIG. 5, also as described above. Waveguides 500 and 520 are separated by a material of refractive index less than that of both waveguides, including but not limited to air, such that light coupled into each waveguide at points 512 and 522 propagates by TIR only in the corresponding waveguide. The patterning and signal separation methods described for previous embodiments may be applied to one or both waveguides. Light emitted from light source 530 is partially or completely absorbed in waveguide 500, possibly in amounts varying over the surface of waveguide 500, therefore the intensity of light incident on waveguides 500 and 520 will differ. However, the difference in incident light intensity is fixed and easily accounted for by modifying the amount of light coupled into each waveguide using the techniques described above. This method of arranging multiple waveguides, not necessarily limited in number to two, such that they partially or completely overlap will be henceforth referred to as “waveguide stacking” and provides a powerful method of increasing the number of independent signal layers in a device.

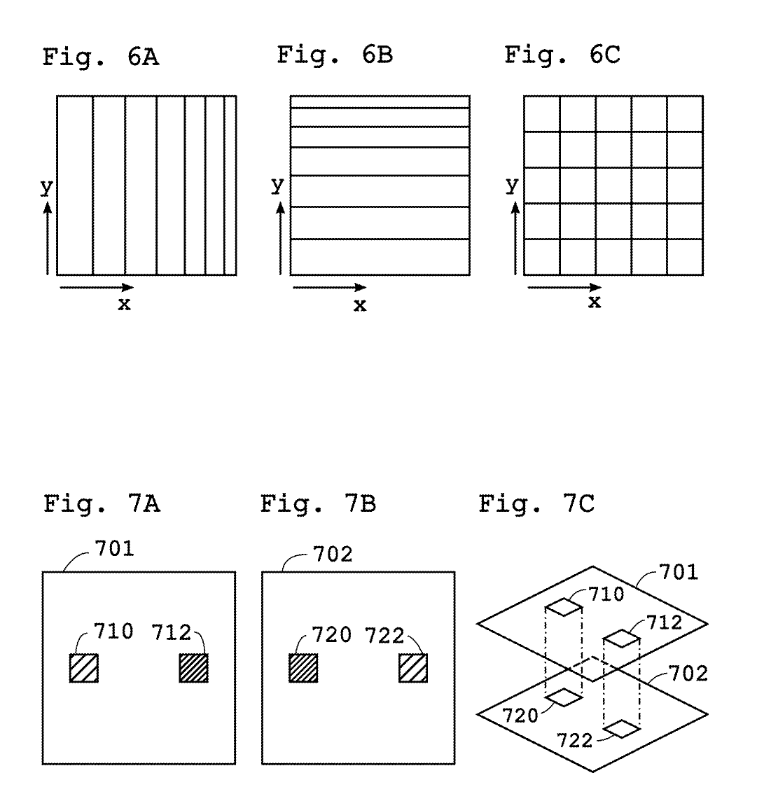

Still another embodiment of the present invention is a device containing three signal layers 601, 602, and 603 which are represented as contour graphs in FIGS. 6A, 6B, and 6C, respectively. The signal layers may, for example, be implemented using three photoluminescent materials of differing emission spectra in a single waveguide and three photosensors each sensitive to the emissions of only one photoluminescent material, as described in a previous embodiment, or any combination of techniques of the present invention. Signal layer 601 varies linearly along the x-axis and is constant along the y-axis of the active area. Signal layer 602 varies linearly along the y-axis and is constant along the x-axis. Signal layer 603 is constant over the entire active area of the device.

As in previous embodiments, light is coupled into the device at a point in the active area of the device at some point of coordinates (x, y). The signal measured from signal layer 601, S_601, is linearly proportional to the amount of light coupled into the device, C_i, and the x coordinate of the point, which may be written mathematically as
S_{—}601=K_{—}601*C_{—}i*x+K_{—}x0
where K_601 is a constant of proportionality and K_x0 is a fixed offset determined by the value of the signal layer at x=0. Similarly, the signal measured from signal layer 602 may be written as
S_{—}602=K_{—}602*C_{—}i*y+K_{—}y0
where y is the unknown y coordinate of the point, K_602 is again a constant of proportionality, and K_y0 is a fixed offset. Finally, because signal layer 603 is of constant value, the measured signal of signal layer 603, S_603, may be expressed as
S_{—}603=K_{—}603*C_{—}i
where K_603 is a constant of proportionality. The constants of proportionality and offsets above are all known and fixed for the system. In the case where K_601, K_602, K_603, K_x0, and K_y0 are all zero, the system of equations above becomes simply:
S_{—}601=C_{—}i*x
S_{—}602=C_{—}i*y
S_{—}603=C_{—}i.

The above equations are trivially solved yielding the coordinates of the unknown point and the amount of light incident at the point. Note that if the amount of light coupled into the device, C_i, is known and fixed, the unknown coordinates (x, y) may be determined using only signal layers 601 and 602, simplifying construction of the device.

A further embodiment contains two signal layers 701 and 702 as illustrated in FIGS. 7A and 7B which cover the active area of the device. Signal layer 701 contains two regions of constant, non-zero value 710 and 712 and is elsewhere zero. Signal layer 702 similarly contains two regions of constant, non-zero value 720 and 722 and is elsewhere zero. As illustrated in FIG. 7C, regions 710 and 720 coincide, as do regions 712 and 722. Note that FIG. 7C is meant to illustrate the coincident arrangement of signal layers; the signal layers may be implemented using any of the techniques of the present invention. As in previous embodiments, light is coupled into the device at a point in the active area at unknown coordinates (x, y). Unlike previous embodiments, however, the present embodiment determines which, if any, non-zero region the unknown point overlaps. Regions 710 and 712 have values of, respectively, a and b. Similarly, regions 720 and 722 have values of, respectively, c and d. When the unknown point falls within the area defined by regions 710 and 720, the measured signals from signal layers 701 and 702, S_701 and S_702, will be proportional to, respectively, the values a and c. This is expressed mathematically as
S_{—}702=C_{—}i*a
S_{—}702=C_{—}i*c
where C_i is the amount of light coupled into the device. Regardless of the amount of light, then, the ratio S_701/S_702 is equal to a/c, which is known and constant. Similarly, when the unknown point falls within the area defined by regions 712 and 722, the ratio S_701/S_702 is equal to b/d. a, b, c, and d are chosen so that the ratios a/c and b/d are unique. This embodiment effectively forms “buttons” which may be simply identified using the ratio of two signal layers in situations with unknown amounts of light coupled into the device. In situations when the amount of light coupled into the device is known and constant, a single signal layer of “buttons” suffices. The number of buttons is limited only by the resolution of the systems used to measure the signals associated with each signal layer.

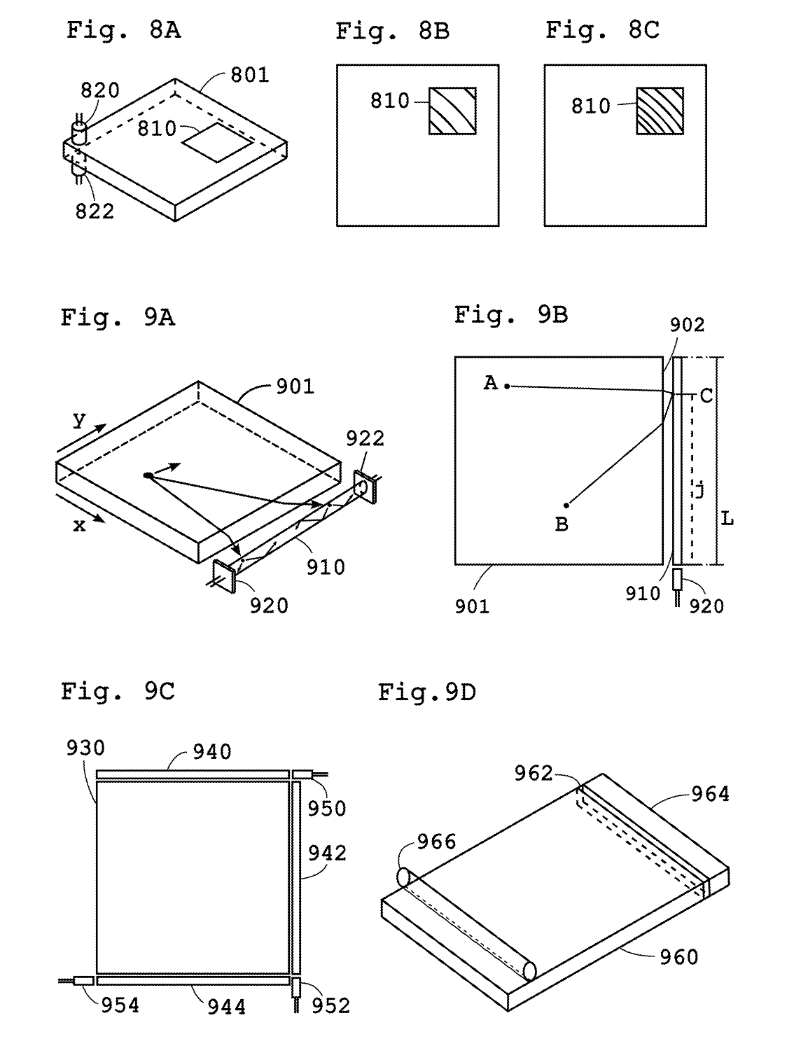

Still another embodiment closely related to the previous embodiment is illustrated in FIG. 8A.

A waveguide 801 is dyed with a first and second photoluminescent dye of differing emission spectra over a region 810. The concentrations of the first and second dyes are constant over region 810 and denoted, respectively, as A and B. Two photosensors 820 and 822 are respectively configured to measure the amounts of light coupled into waveguide 801 by the first and second dyes, as described for a previous embodiment. The waveguide-photosensor responses, or signal layers, for the system are shown in FIGS. 8B and 8C. The signal layers are not constant over region 810, unlike the previous embodiment, because of factors including the spreading and attenuation of light as it propagates within waveguide 801, as described above. However, for any point in region 810 the ratio of values is constant and so, as in the previous embodiment, buttons may be simply identified as unique, fixed ratios of signal levels regardless of variations in the amount of light coupled into the device.

Yet another embodiment is illustrated in FIG. 9A. As before light is coupled into a waveguide 901 at an unknown point of coordinates (x, y) in a Cartesian plane containing waveguide 901. Light coupled into waveguide 901 propagates until reaching an edge where it exits and strikes a light collecting element (LCE) 910. LCE 910 forms an essentially one-dimensional waveguide. Light incident on the surface of LCE 910 is partially or completely coupled into LCE 910 by any of the previously mentioned techniques including photoluminescence and surface roughening. Light coupled into LCE 910 travels along the element until reaching an end where it is measured by one of photosensors 920 or 922. Light propagating along LCE 910 is subject to various phenomena including scattering and absorption as described previously, so the amount of light reaching a photosensor after being coupled into LCE 910 at a point on its surface will in general depend on the position of the point or, equivalently, the separation distance between the point and the photosensor. It will be apparent to a skilled practitioner that this relationship is just a waveguide-photosensor response as described above, and will henceforth be referred to as a LCE-photosensor response for purposes of distinction.

Referring now to FIG. 9B, the relationship between the unknown point location and the signal produced by each photosensor will now be described. The distance between LCE 910 and waveguide 901 has been exaggerated in FIG. 9B and will be assumed to be zero for practical purposes. Light coupled into waveguide 901 at the unknown point propagates until reaching an edge 902 where it exits and strikes LCE 910. Upon striking LCE 910 some light is reflected and some is transmitted into LCE 910 where it is partially or completely coupled to propagate along the length of LCE 910 by TIR. The amount of light coupled into LCE 910 to propagate by TIR is dependent on several factors including the angle at which it is incident on the surface of LCE 910 and so will in general depend on the location of the unknown point in waveguide 901. Consider the two positions in waveguide 901 A and B, and a location C on LCE 910 as shown in FIG. 9B. When the unknown point is located at position A the light striking LCE 910 at location C is nearly normal to the long axis of LCE 910. When the unknown point is located at position B, however, the light striking LCE 910 at location C is at a much shallower angle to the surface and less light will be coupled into LCE 910, all other factors being equal. The amount of light coupled into LCE 910 at a point of coordinate (j) therefore depends on the location of the unknown point, (x, y), which may be written as C(j, x, y). Note that other factors dependent on the location of the unknown point in waveguide 901 including separation distance also play parts in determining C(j, x, y).

The total amount of light reaching a photosensor is just the sum of the amounts of light coupled into LCE 910 at all points on its surface, modified by the LCE-photosensor response LP, or mathematically
S=integral_over_{—}L(LP(j)*C(j, x, y)*dj),
where S is the measured signal output by the photosensor. The relationships LP(j) and C(j, x, y) may be determined using any number of techniques familiar to a skilled practitioner including numerical analysis and direct measurement, as described in a previous embodiment.

The determination of the coordinates of the unknown point proceeds as above, forming a system of equations relating the location of the unknown point (x, y) to the measured signals from an appropriate number of photosensors, S_i, and solving for the point (x, y). Multiple photosensors may be associated with a single LCE, as shown in FIG. 9A, or a single photosensor may be coupled to the LCE. Multiple LCEs may be used, one for each edge, or multiple LCEs may be provided for a single edge. Consider the arrangement shown in FIG. 9C. Three LCEs of length L, 940, 942, and 944 are coupled to three photosensors 950, 952, and 954 as shown. The three LCE-photosensor units are arranged to receive light escaping the edges of a waveguide 930 as shown. The system of equations relating the (x, y) position of a point on waveguide 930 where light is coupled into the waveguide to the output signal of a photosensor p, S_p, is:
S_{—}950=C_{—}i*integral_over_{—}L(LP_{—}950(j)*C_{—}940(j, x, y)*dj)
S_{—}952=C_{—}i*integral_over_{—}L(LP_{—}952(j)*C_{—}942(j, x, y)*dj)
S_{—}954=C_{—}i*integral_over_{—}L(LP_{—}954(j)*C_{—}944(j, x, y)*dj)
where C_i is the amount of light coupled into the device and L is the length of each LCE.

The LCE-photosensor responses may be modified using waveguide patterning techniques described above. Similarly, other techniques described above such as those for waveguide multiplexing are applicable to LCEs as well.

FIG. 9D shows several alternative LCE arrangements. A waveguide 960 is coupled to two LCEs 964 and 966. LCE 964 has a square cross-section and is not coupled to waveguide 960 directly but rather through a filter 962 made of a material that passes light of wavelengths in the excitation spectrum of LCE 964 but absorbs light of wavelengths in the emission spectrum of LCE 962. In this manner crosstalk is prevented because light emitted by PL in LCE 964 is not propagated through waveguide 960 where it might eventually strike photosensors associated with other LCEs in the system. LCEs need not be coupled to a waveguide at an edge. In some instances it is convenient to attach a LCE to the “top” or “bottom” of a waveguide, as in the case of LCE 966. LCE 966 is composed of a material of suitable refractive index and attached to waveguide 960 such that some light propagating in waveguide 960 by TIR enters LCE 966. LCE 966 might, for example, be made of a the same base polymer and, hence, refractive index as waveguide 960. This method of attachment permits the coupling of two LCEs at the same location in the plane of a waveguide, one on each “face” of the waveguide.

Note that the discussions above have been simplified for explanatory purposes. The geometry and materials used to implement a particular embodiment will determine the exact relationships involved, as will be apparent to a skilled practitioner.

Embodiments have been described above that determine the location of light incident at an unknown point on the active area of a device. It is often desirable to track or determine the locations of multiple unknown points simultaneously. Multiple points may be tracked simultaneously using the methods described above simply by increasing the number of signal layers and solving the resulting system of equations using the measured signal values. In general when the amount of light incident at each point is known, at least two additional signal layers are necessary for each additional tracked point. When the amount of incident light at each point is unknown, at least three additional signal layers are necessary for each addition point to be tracked. Still more signal layers may be required depending on the geometry of the active area and the placement of photosensors, as will be apparent to a skilled practitioner.

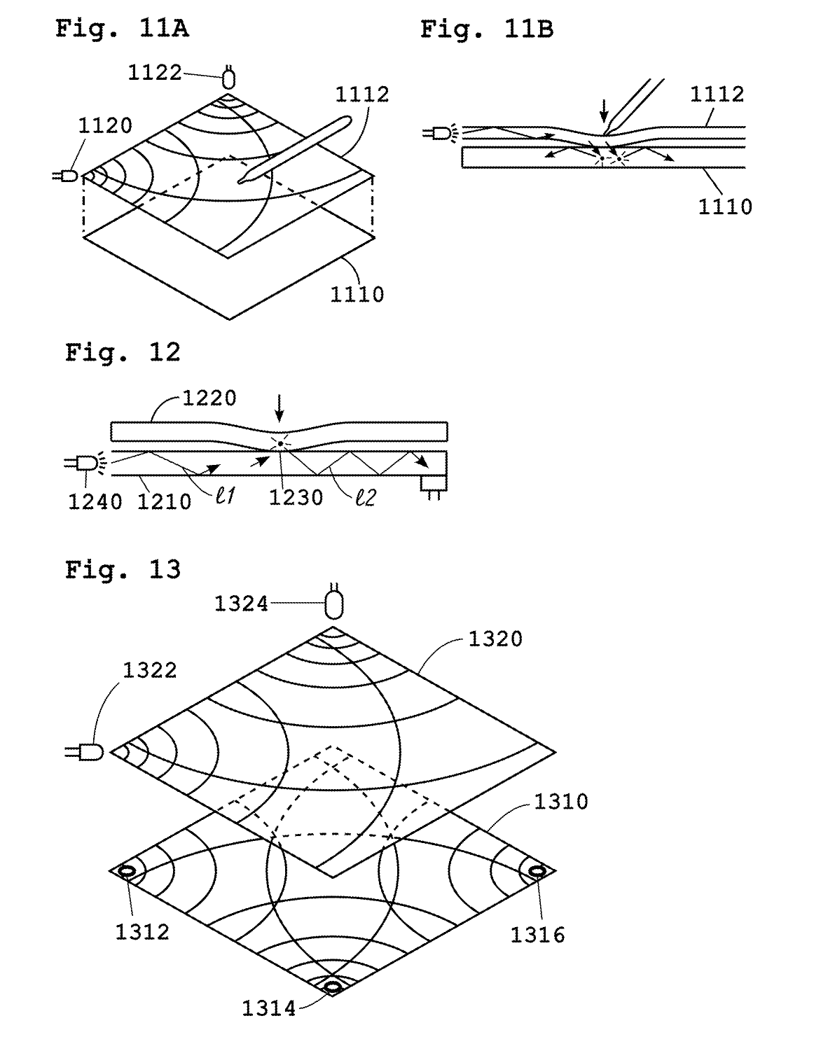

Still another embodiment converts pressure at a point on the active area of a device to light incident at the point on a waveguide or waveguides of the device. FIG. 11A shows a device containing a sensing waveguide 1110 constructed according to techniques of previous embodiments and a waveguide 1112 configured proximal to waveguide 1110. Two light sources 1120 and 1122 are configured to inject light of a spectrum to which sensing waveguide 1110 is responsive into waveguide 1112 where it propagates by TIR. A waveguide propagating stimulating light will henceforth be referred to as a stimulating waveguide. Stimulating waveguide 1112 and sensing waveguide 1110 are separated by a medium of refractive index lower than that of both waveguides. Stimulating waveguide 1112 is composed of a flexible material such that when a downward force is applied at a point on its surface as illustrated in FIG. 11B, stimulating waveguide 1112 and sensing waveguide 1110 are brought into contact. The greater the downward force, the greater the area of contact. Waveguides 1110 and 1112 have similar refractive indices such that light propagating in stimulating waveguide 1112 is coupled into waveguide 1110 at the area of contact. The amount of light coupled into sensing waveguide 1110 is proportional to the applied pressure. The position of this point of contact and amount of light and therefore pressure is then determined according to techniques described above.

The two waveguides are configured such that an certain amount of pressure at a point on the surface of stimulating waveguide 1112 causes a certain amount of light to be coupled into sensing waveguide 1110 which is the same for all points on the active area of the device. In this case stimulating waveguide 1112 is said to have a constant light pressure for all points, or simply a constant light pressure distribution.

A further embodiment reverses the arrangement of the previous embodiment such that a force normal to the plane of the device applied at a point on the sensing waveguide deforms the sensing waveguide such that it contacts the stimulating waveguide.

A side view of yet another embodiment is partially shown in FIG. 12. A light source 1240 emits light l1 which propagates along a waveguide 1210 until reaching a point 1230. A downward force causes an upper transparent, photoluminescent layer 1220 into contact with waveguide 1210 at point 1230. Light l1 enters layer 1220 at point 1230 where it induces photoluminescence in layer 1220, causing light 12 to be emitted. Some of light 12 enters waveguide 1210 and propagates by TIR until reaching photosensors sensitive only to parts of the spectrum of light 12 not present in the spectrum of light l1, as described in previous embodiments. In this way upper layer 1220 acts as a spectrum-shifting, diffuse reflector creating a light source located at point 1230 which may be differentiated from the stronger, stimulating light l1. In this case waveguide 1210 acts as both stimulating and sensing waveguide. Both waveguide 1210 and layer 1220 may be constructed using materials transparent to visible light, including those described above for previous embodiments. Layer 1220 may alternatively be opaque and photoluminescent, constructed for example using many commonly available inorganic photoluminescent pigments. In this case light l1 is absorbed by the photoluminescent material in layer 1220 when it is forced into contact with waveguide 1210 and some emitted light is coupled into waveguide 1210. The position of the contact point is tracked by any appropriate method of the present invention. For those methods which require the absorption of light coupled into the sensing waveguide at its edges the edges may be treated to absorb light l2 and optionally reflect light l1.

A further embodiment shown in FIG. 13 contains a stimulating waveguide 1320 and a sensing waveguide 1310 coupled to three photosensors 1312, 1314, and 1316. Two light sources 1322 and 1324 couple light into stimulating waveguide 1310 which is then coupled at an unknown point or points into sensing waveguide 1310. The positions of the unknown point or points are determined by any of the methods of the previous embodiments using measured output signals of photosensors 1312, 1314, and 1316. This configuration is similar to that of the system in FIG. 11A except that the light pressure distribution of stimulating waveguide 1320 is not constant over its surface. The concept of a signal layer was introduced in the embodiment of FIG. 3A and 3B to refer to a mapping of light incident at a point on the surface of the device to the resulting photosensor output signal. This term will be applied to the current embodiment and other pressure-sensing configurations to mean a mapping of pressure at a point on the active area of the device to the resulting photosensor output signal.

FIG. 13 shows the light pressure distributions associated with each of light sources 1322 and 1324 as contour lines on stimulating waveguide 1320. It may be intuitively seen, for example, that a given amount of pressure at a point close to light source 1322 will couple more light from light source 1322 into sensing waveguide 1310 than the same amount of pressure applied at a point further from light source 1322. The amount of light coupled into sensing waveguide 1310 in turn affects the photosensor output signals. Therefore, a signal layer in the system of FIG. 13 is determined by both a waveguide-photosensor response and a light pressure distribution in the stimulating waveguide. Light sources 1322 and 1324 are modulated independently in a way such that their contributions to each photosensor output signal is distinguishable, such as by carrier modulation or multiplexing in time. Each photosensor is therefore associated with two signal layers, one for each light source. The system provides a total of six signal layers using three photosensors and two light sources, enabling the tracking of up to two independent points simultaneously. This technique is useful for increasing the number of signal layers in situations when it is undesirable to increase the number of photosensors. This configuration is shown with two light sources and three photosensors, but it will be clear to the skilled practitioner that the number of light sources and/or photosensors may be adjusted to allow the simultaneous tracking of a desired number of contact points.

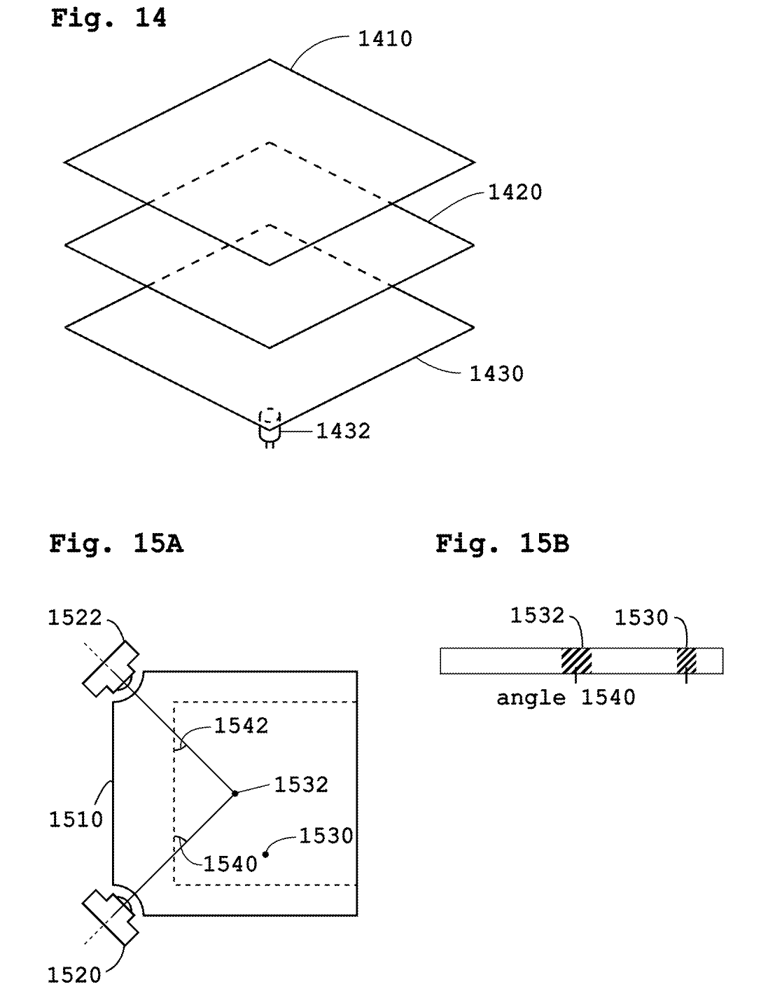

FIG. 14 shows still another embodiment. A stimulating waveguide 1410 carrying light l1 is arranged proximal to a filter layer 1420 and a sensing waveguide 1430 as shown. A downward force is applied forcing the three layers into contact at a point to be tracked. Unlike previous pressure-sensing embodiments, however, light from stimulating waveguide 1410 passes through filter layer 1420 before entering sensing waveguide 1430. Filter layer 1420 is patterned using three different dyes or dopants to control the amounts of three different regions of the spectrum of light l1 passed into sensing waveguide 1430. Light coupled into waveguide 1430 propagates within the waveguide, some of which reaches a photosensor 1432. Photosensor 1432 has three outputs each corresponding to one of the three parts of the spectrum of light l1 modulated by filter layer 1420. Filter layer 1420 is patterned as described in the embodiment of FIGS. 6A, 6B, and 6C such that the pressure and position of the unknown point are easily determined. The edges of stimulating waveguide 1410 and/or sensing waveguide 1430 may be mirrored to improve light efficiency.

Further embodiments use the filter layer approach of the previous embodiment to effectively pattern the sensing waveguide.

Still further embodiments include a diffusing layer between a stimulating and a sensing waveguide, or between a dual-purpose waveguide and a reflecting layer. When a normal force is applied at a point on the active area all layers are forced into contact and the diffusing layer acts to diffuse light coupled into the waveguide(s). The diffusing layer may be composed of any material with appropriate scattering properties. The diffusing layer may act to diffuse light of only certain wavelengths, including invisible portions of the spectrum. For example, small particles of titanium dioxide such as those used in the manufacture of sunscreen could be embedded in a transparent polymer host material to scatter ultraviolet wavelengths while passing visible wavelengths largely unchanged creating a device transparent to visible light.

Still another embodiment is similar to the embodiment of FIG. 14 in that it contains a stimulating waveguide, a filter layer, and a sensing waveguide. Three light sources inject light of three different spectra into the stimulating waveguide. The filter layer is patterned as in the embodiment of FIG. 14 to modulate at each point on the active area the amount of light from each light source coupled into the sensing waveguide. The sensing waveguide is coupled to a single photosensor which produces an output signal proportional to the sum of the amount of incident light from each light source. Each light source is modulated independently such that part of the output signal due to each light source may be separated using signal processing techniques. This embodiment achieves three signal layers using three light sources and a single photosensor. The position and pressure of the unknown point is determined as in the previous embodiment and the embodiment of FIGS. 6A, 6B, and 6C.

Yet another embodiment is illustrated in FIG. 15A. A waveguide 1510 is provided with an active area indicated by dashed lines. The edges of waveguide 1510 are treated to suppress reflections. Two circular regions have been cut out of waveguide 1510 so that light propagating by TIR within the waveguide can escape and be measured by two imaging systems 1520 and 1522. The imaging systems may contain simple one-dimensional image sensors such as CCDs or photodiode arrays, or two-dimensional sensors. Light is coupled into waveguide 1510 at a point or points in the active area by any of the methods described above, including photoluminescence and scattering. Light incident on waveguide 1510 may come from a distant light source or sources, be coupled by pressure on a second waveguide not shown, or any other suitable method. FIG. 15B illustrates the output of imaging system 1520 for two cases when light is coupled into waveguide 1510 at point 1532 or 1530. It may be intuitively understood that the location of the image of the unknown point in the output of imaging system 1520 depends on an angle 1540 as shown in FIG. 15A. The exact relationship between angle 1540 and the position in the output image of imaging system 1520 depends on the optics used and is easily determined. Likewise an angle 1542 is easily determined from the output of imaging system 1522. These two angles, together with simple geometry, suffice to uniquely determine the position of the unknown point. If the imaging systems provide intensity information, the amount of incident light or, where appropriate, pressure, is trivially determined. This configuration can always uniquely determine the position of a single unknown point. More points may be simultaneously determined in some instances, but only when all points are visible to both imaging systems, i.e., when there is no occlusion. Adding more imaging systems increases the number of unknown positions that can be reliably determined or tracked.

Still another embodiment uses multiple imaging systems as described in the previous embodiment to track multiple points. Each imaging system measures not only incident light intensity as a function of incident angle, but also spectral information such as RGB color. Methods described above such as filter layers and/or waveguide patterning are used to spatially vary the spectral content or color of light coupled into a sensing waveguide. This color information is then used to correlate the unknown point images in the outputs of the imaging systems. A concrete example follows as an aid to understanding. A color filter is arranged between a sensing waveguide and a waveguide carrying white-light. At a point A the filter passes only blue light and at a point B the filter passes only red light. Three imaging systems are provided. When pressure is applied at both points, the image of light coupled at point A is blue and the image of light coupled at point B is red in the outputs of all imaging systems. It is clear which point in each output is from point A and which from point B.

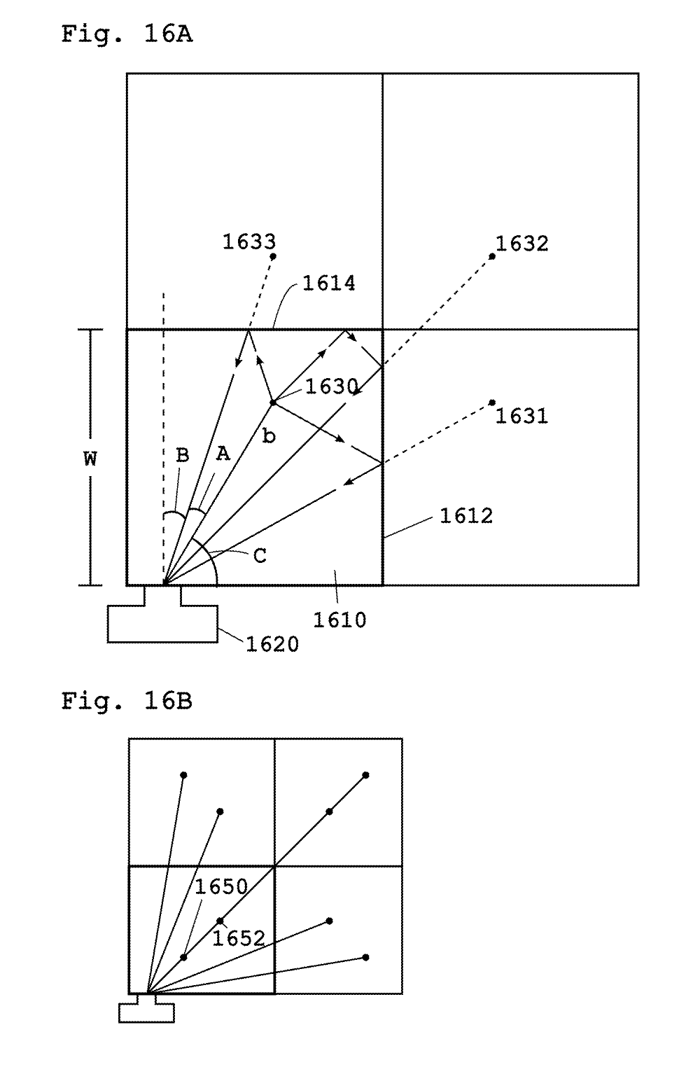

A further embodiment is presented in FIG. 16A in a top view. An imaging system 1620 is configured to provide a one-dimensional color output image of light coupled into a waveguide 1610 at up to two unknown points. Side 1612 of waveguide 1610 is treated to reflect a part S1 of the spectrum of light coupled into waveguide 1610, and a side 1614 is treated to reflect a different part S2 of the spectrum of light coupled into waveguide 1610. The remaining two sides of waveguide 1610 are treated to suppress reflections. An unknown point 1630 located a distance b from imaging system 1620 is shown along with its reflections 1631, 1632, and 1633 and reflections of waveguide 1610. Point 1630 forms angle A with its reflection point 1633 and angle C with a waveguide edge parallel to edge 1614. Point 1633 forms an angle B with a waveguide edge parallel to edge 1612. Angles A, B, and C are easily determined from the output of imaging system 1620, noting that the images of points 1633 and 1630 always appear left to right in that order. The images of points 1633 and 1630 may also be distinguished by color due to the nature of edge 1614. Given the angles A, B, and C, the distance b is given by the formula:
b=(2*w*sin (B))/(2*sin (B)*sin (C)+sin (A))
where w is the length of a side of waveguide 1610. Angle C and distance b uniquely determine the location of point 1630.

Note that reflections 1631 and 1632 were not used in the above determination of the location of point 1630. It order to determine the location of an unknown point, it is in general sufficient to measure the directions of any two of the point and its three reflections. The worst case when tracking two points is shown in FIG. 16B. A point 1650 occludes a second point 1652 along with two reflections, but two reflections of each point 1650 and 1652 are still visible to the imaging system therefore providing enough information to determine the location of each point. The system of FIG. 16A may therefore track at least two points simultaneously. The formulas for determining the location of an unknown point based on the directions of its various reflections are easily derived using simple geometry and so not provided here.

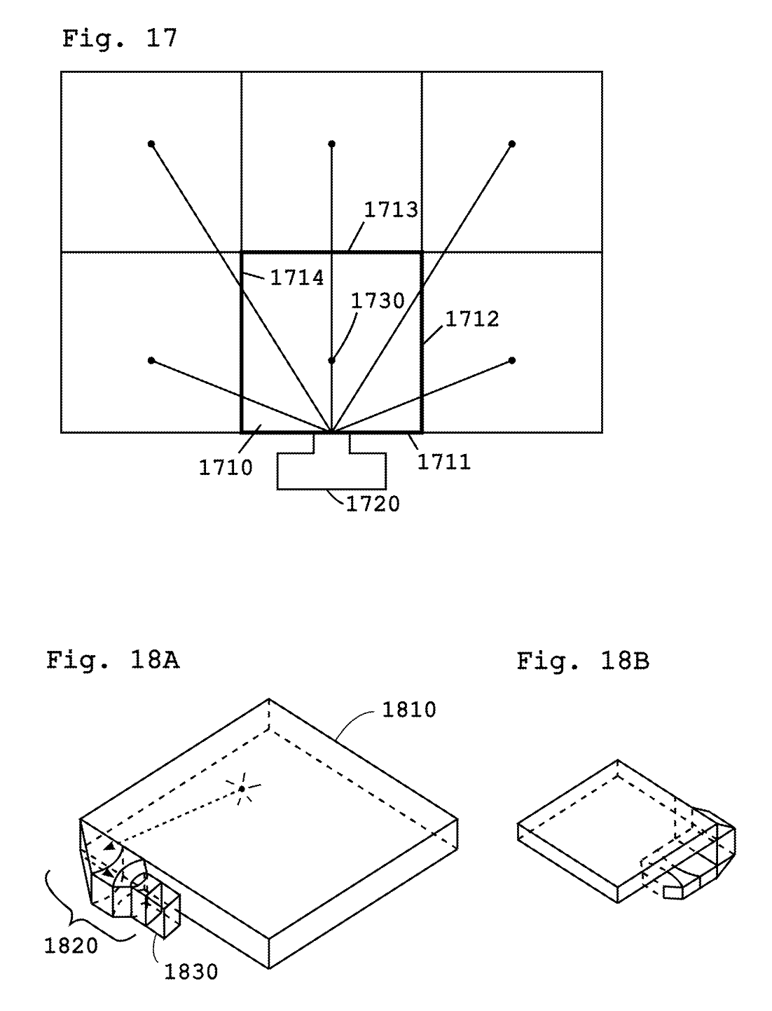

FIG. 17 shows a further embodiment. An imaging system 1720 is configured to view the active area of a waveguide 1710. Light is coupled into waveguide 1710 by any suitable means at an unknown point 1730. Five reflections of point 1730 are shown. A side 1711 of waveguide 1710 is treated to suppress reflections. Three remaining sides 1712, 1713, and 1714 are treated to reflect three parts S2, S3, and S4 of the spectrum of light coupled into waveguide 1710, respectively. Spectra S2 and S3 partially overlap, as do spectra S4 and S3. Spectra S2 and S4 do not overlap at all to prevent a nearly infinite number of reflected point images which would make signal processing difficult. For example, if white light is coupled into waveguide 1710, spectrum S3 might contain only red and blue while spectrum S2 contains only red and spectrum S4 contains only blue. Imaging system 1720 provides spectral information for each image and spectra S2, S3, and S4 are chosen such that point 1730 and each reflection may be distinguished on the basis of spectral content alone. This system can track at least four points simultaneously. The position of each tracked point is determined as described in the previous embodiment by finding for each point at least two images of any of the point and its reflections and then computing the distance from the imaging system.

A further embodiment is similar to the previous embodiment except that the imaging system does not provide spectral information per se. Instead, light coupled into the sensing waveguide is composed of three different spectral components produced by three individually modulated light sources. Spectral information is determined from the output of the imaging system using demodulation/demultiplexing techniques as described for previous embodiments.

Still another embodiment is shown in FIG. 18A. A waveguide 1810 propagates light via TIR to an optical element group 1820 which produces an image on an image sensor 1830. Optical element group 1820 is similar in thickness to waveguide 1810 and composed of materials of varying refractive index bonded to each other and the waveguide to form a thin optical path that can easily fit in a device where space is at a premium. FIG. 18B shows an alternative arrangement where the optical path is folded under the transparent active area of the device leaving a space or slot where a display such as an LCD panel might be placed.

Additional embodiments use more than one imaging system to decrease the potential for occlusion and thus increase the minimum number of points which may be reliably tracked.

In the discussion above, the term “occlusion” is used to describe the condition where two or more images overlap as seen from a given point of view. In the cases above, however, it is to be understood that because the images are not physical objects there is no actual “occlusion” but rather an addition of the overlapping images.

Further embodiments use imaging systems such as those described above to distinguish different sizes, orientations, and patterns of light coupled into sensing waveguides by comparing the images of the contact areas and their reflections with a database shape, size, and pattern information.

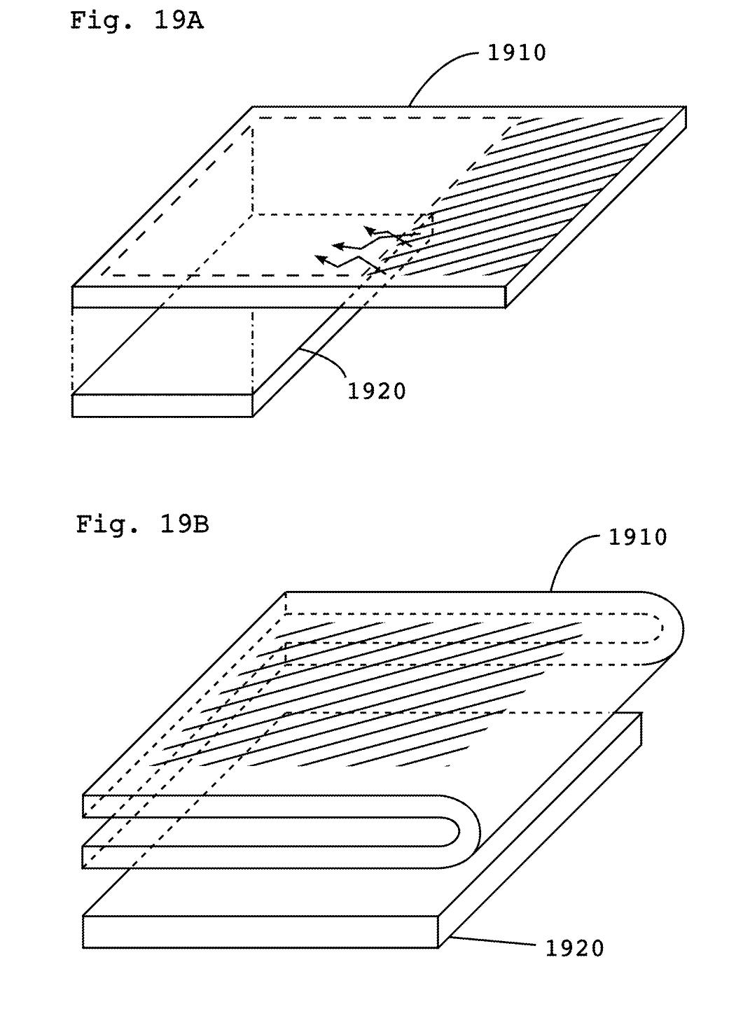

A further embodiment is illustrated in FIG. 19A. An upper stimulating waveguide 1910 is positioned proximal to a lower sensing waveguide 1920 as shown. The shaded region of stimulating waveguide 1910 is doped with photoluminescent material excited by ambient light of a first spectrum S1 which emits light of a second spectrum S2. Part of the light of spectrum S2 is trapped in waveguide 1910 to propagate by TIR. The edges of waveguide 1910 are mirrored, trapping light of spectrum S2 inside. Light of spectrum S2 travels through the active area of the device denoted by dashed lines. When a downward force is applied at a point or points on the active area it is forced into contact with sensing waveguide 1920 as described in previous embodiments. Light of spectrum S2 travels into sensing waveguide 1920 at the point of contact. The location of the point of contact is determined using any of the methods of the previous embodiments. If light of spectrum S2 is present in the environment additional shield layers are placed as necessary above and below waveguides 1910 and 1920 to prevent light of spectrum S2 from the environment from striking the active area of the device. By using light from the environment power requirements may be reduced in situations when power is at a premium. Light sources producing light of spectrum S2 may be coupled to waveguide 1910 and activated in situations where ambient light is not sufficient for sensing. FIG. 19B illustrates an alternative configuration where the ambient light conversion region is folded over top of the undoped region both saving space and acting as a shield preventing light of spectrum S2 from the environment from striking sensing waveguide 1920.

Still further embodiments combine ambient light conversion with filter layers, multiple dopants producing distinct spectra of light, waveguide patterning, or any other techniques of previous embodiments.

Still other pressure-sensing embodiments include a layer of material between two surfaces which are to be optically coupled by the application of a normal force. Such a “coupling layer” may be composed of a soft material with an affinity for both surfaces to be coupled. Polyolefin elastomers are one such suitable material. This approach is useful when the material of the coupling layer would adversely affect the propagation of light within a waveguide were the coupling layer bonded directly to the waveguide. The coupling layer may simultaneously serve as a diffusing and/or filter layer as described previously.

Still other embodiments attach optical fibers or other optical channels at points on a waveguide where light is to be measured to carry the light to remotely-mounted photosensors. Since optical fibers are not sensitive to electromagnetic interference (EMI) this technique is useful in noisy environments.

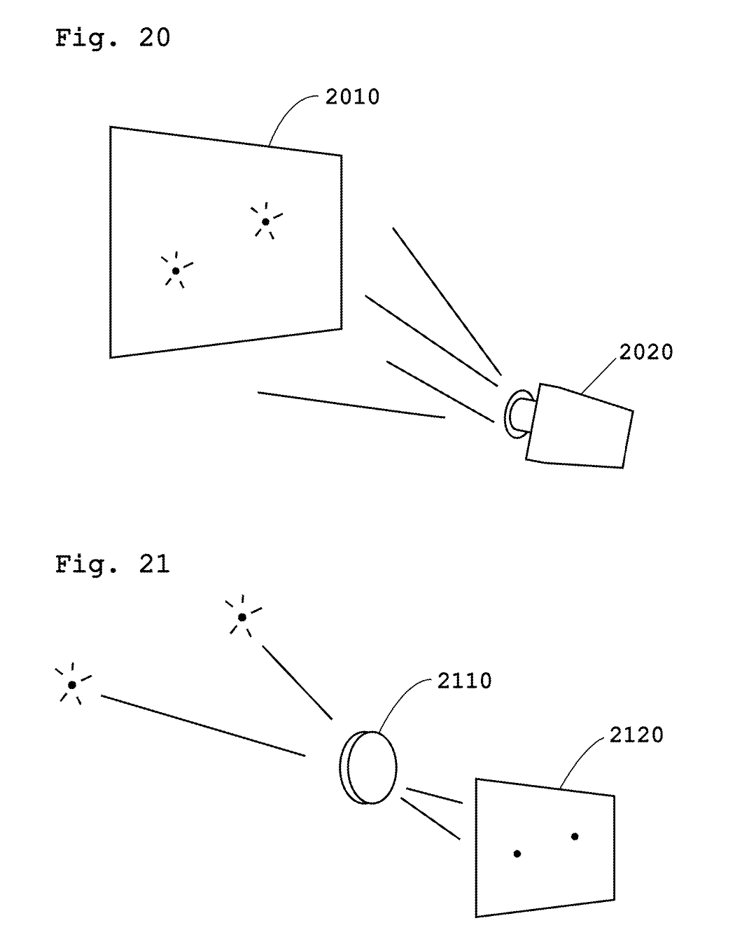

Still further embodiments apply the techniques of previous embodiments to create systems of the form illustrated in FIG. 20. Diffuse point sources of light to be sensed are created in an interaction surface 2010. Note that many of the techniques presented in the previous embodiments effectively form diffuse point sources of light, some of the light escaping the associated system of waveguides so that it may be observed from a remote location. An imaging system 2020 such as a camera captures an image of the interaction surface and computer vision techniques are used to track the point sources of light. In particular, the techniques presented in this document permit the construction of an interaction surface transparent to visible light, something not previously possible. Further details may be found in the patent application of Tulbert mentioned above.

Still other embodiments add to the systems presented above an optical imaging system to track distant objects, as illustrated in FIG. 21. An optical imaging system 2110 such as an optical lens system projects an image of distant light sources onto a sensing device 2120 constructed according to previous embodiments. The sensing devices presented in this document are well-suited to highly accurate, high-speed, low-cost, simultaneous tracking of multiple objects.

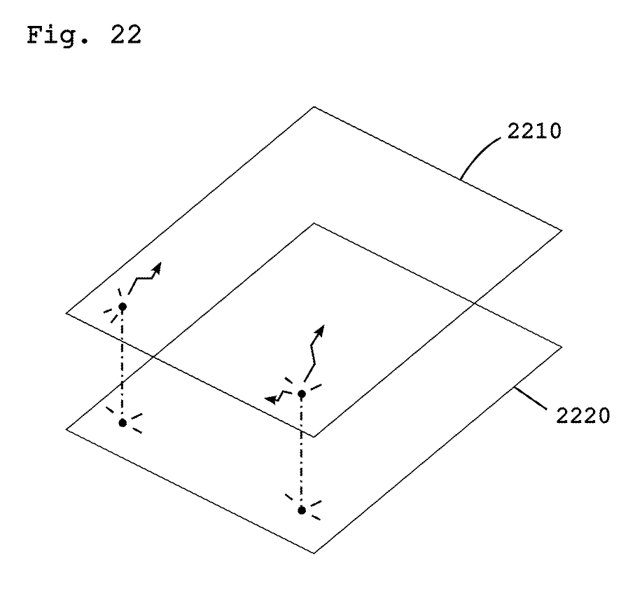

FIG. 22 shows an additional embodiment. A transparent sensing surface 2210 constructed according to previous embodiments is positioned above a light emitting display device 2220. A common problem in the field of touch-responsive displays is the calibration or registration of the touch-sensing device and the display device. Because the methods of this invention are optical in nature, the display device can itself be used to generate calibration signals. Display device 2220 displays a known test pattern in wavelengths of light to which sensing surface 2210 is sensitive. The output signals of sensing surface 2210 are compared with stored values and used to correct for any misalignment. For example, if display device 2220 is a LCD, a calibration mode of the backlight emits light to which sensing surface 2210 is sensitive. Several points are displayed on the screen sequentially, coupling light into corresponding points on sensing surface 2210. The points are tracked and the tracked positions are compared to the point positions on display device 2220, yielding the desired information on any misalignment of sensing surface 2210 and display device 2220.

Yet another embodiment of the current invention comprises one or more photoluminescent waveguides and photosensors having more than one signal layer and corresponding output signal. In this case the output signals are not used to create a system of equations, but are used rather to reconstruct an approximation of the distribution of light incident on the waveguide(s). This is accomplished by graphing the sum of all waveguide-photosensor response functions where each response function is scaled by the corresponding photosensor output signal. For example, signal separation methods described above may be used to create signal layers that form a set of small, closely-spaced, non-overlapping squares covering the common plane of the waveguide(s). In this case each response function corresponds to a pixel in a conventional imaging sensor. The graph of the sum of the scaled response functions is an image of incident light resembling the output of a conventional imaging sensor such as a CCD. In combination with a lens, this system forms a camera that can be transparent to wavelengths of light not in the excitation spectra of the waveguide(s). Alternatively, the signal layers may be the basis functions of transforms including the Fourier transform, the discrete cosine transform, and various wavelet transforms. In this case the photosensor output signals correspond to the coefficients resulting from the associated transform; in this manner a complicated transform may be performed virtually instantaneously.

Still further embodiments add protective layers to protect waveguides from physical damage and contamination by materials including dust, dirt, and oil, as is common practice in the manufacture of optical systems.

The above embodiments describe many techniques which may be combined in many different ways which will be obvious to one skilled in the art. The embodiments described here are not intended to limit in any way the scope of the invention. In particular, in order to simultaneously track multiple points using non-imaging photosensors, it is sufficient to provide a number of signal layers equivalent to the number of degrees of freedom (two or three per tracked point, depending on the configuration) in the system using any combination of techniques described in this document.

Many of the embodiments described above are generally intended to be implemented using materials transparent to visible light, but any application that does not require transparency in the visible spectrum need only use waveguide materials transparent to the wavelengths of light they are required to propagate.

Note also that although various embodiments have been described and illustrated as planes any geometry through which emitted light may propagate by TIR is possible. Additionally, although visible and ultraviolet light have often been used as examples, near infrared (NIR) light may also be used.

The term “total internal reflection” is often used in the preceding paragraphs to describe the propagation of light in a waveguide, however it is understood that for suitably short distances propagation by specular reflection, i.e. when the propagated light is not totally reflected, is also acceptable.

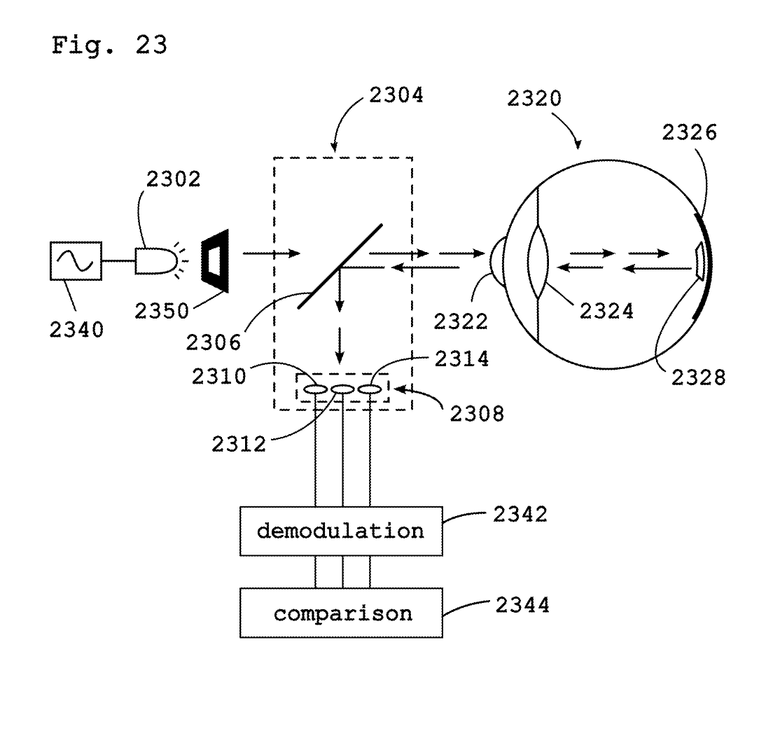

A preferred embodiment of the gaze tracker of the present invention is illustrated in FIG. 23. A modulation element 2340 modulates light emitted from a probe element 2302, which forms a probe pattern 2350. A light sensing element 2304 is provided, comprising a beamsplitter 2306 and a light sensor group 2308. Light sensor group 2308 further comprises three light sensors 2310, 2312, and 2314. Beamsplitter 2306 and light sensor group 2308 are positioned such that, when viewed by an eye 2320, an image of light sensor group 2308 in beamsplitter 2306 appears at the same location as probe pattern 2350. Signals from light sensor group 2308 are processed by a demodulation element 2342, the output of which is then processed by a comparison element 2344.

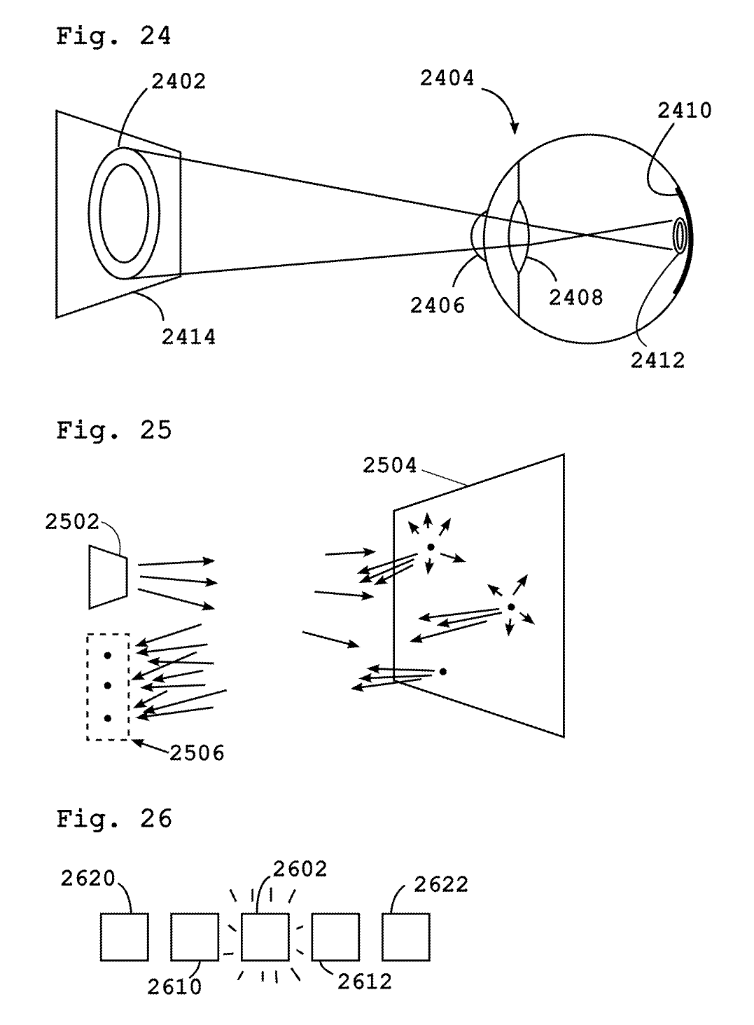

FIG. 24 illustrates a principle central to the operation of the present gaze-tracking invention. An eye 2404 viewing an object 2402 focuses light to create an image 2412 on a retina 2410. While most of the light is absorbed and sent as electrical signals to the brain, some is reflected. Some of the reflected light travels out of eye 2404 through a lens 2408 and a cornea 2406, which focus the light to form an image at a focal plane 2414. Focal plane 2414 is normal to the gaze direction of eye 2404 and contains the point of fixation, which by definition is located somewhere on object 202. Put another way, when a viewer looks at an object, the images formed on the viewer's retinas are re-projected “on top” of the object. It should be noted that this effect occurs not only in the human eye but in any imaging system forming an image at or near a partially reflective surface. This effect occurs in most cameras, for example. The principle illustrated in FIG. 24 is well known and documented, for example in U.S. Pat. No. 5,684,561 to Yancey, which is incorporated herein by reference.

The operation of an embodiment of the present gaze tracking device will now be described with reference to FIG. 23. The embodiment of FIG. 23 is operated at a distance preferably greater than 10 cm from objects in the environment that cause light emitted from probe element 102 to be reflected towards the device. The preferred embodiment is further operated with a viewer or viewers preferably located further than 10 cm and closer than 10 m from the device.

Referring again to FIG. 23, modulation element 2340 provides a signal to modulate in time the intensity of light emitted by probe element 2302 forming probe pattern 2350. The modulation may be of any type suitable for distinguishing light originating from probe element 2302 from that originating from other light sources in the environment of use. However, the modulation is preferably of a low duty cycle type so that a high intensity of light can be emitted without overheating probe element 2302 or damaging a viewer's eyes. Probe pattern 2350 may be any pattern unlikely to be caused by non-image-forming reflections of light in the environment; however, a square of dimension 0.1-5 mm surrounded by a dark border of thickness 0.1-5 mm is currently preferred as many commonly available light emitting diodes (LEDs) form this image.

Light forming probe pattern 2350 may be of any spectral composition but is preferably composed of near infrared (NIR) light in the range 850 to 1000 nm. The pigmented tissues of the retina reflect more light at these wavelengths than in the visible region resulting in a strong reflection that is easy to measure. Also inexpensive NIR light sensors and emitters are commonly available. In addition, many inexpensive optical filters are available that pass the NIR reflections to be measured but block much extraneous light from unrelated light sources.

A viewer's eye 2320 is shown fixated on probe pattern 2350. Light forming probe pattern 2350 is focused by cornea 2322 and lens 2324 to form image 2328 of probe pattern 2350 at retina 2326. Some of the light is reflected out of eye 2320 and is focused by lens 2324 and cornea 2322. As explained above and illustrated in FIG. 24, light exiting eye 2320 forms an image of image 2328 at focal plane containing probe pattern 2350. In effect, eye 2320 projects an image of probe pattern 2350 to form at the location of probe pattern 2350.

Part of the projected light is reflected by beamsplitter 2306 towards light sensor group 2308. Because light sensor group 2308 and probe pattern 2350 are equidistant from beamsplitter 2306, the projected light forms an image at light sensor group 2308. Light sensors 2310, 2312, and 2314 are preferably photodiodes with spectral responses matched to the spectral composition of probe pattern 2350. However, the light sensors may be of any type with appropriate characteristics, for example a charge-coupled device (CCD) or CMOS imaging sensor of the type used in consumer video cameras.

The location of light sensor 2312 corresponds to the center of probe pattern 2350, and its size is equal to or slightly greater than the size of the square in probe pattern 2350. The locations of light sensors 2310 and 2314 correspond to points in the black border of probe pattern 2350. Light sensors 2310 and 2314 are both preferably of the same size as light sensor 2312. Therefore, the focused light exiting eye 2320 and forming an image of probe pattern 2350 at light sensor group 2308 strikes light sensor 2312, but not light sensor 2310 or 2314 since each corresponds to a dark part of probe pattern 2350.

Light striking light sensor group 2308, and thus the resulting output signals, may be divided into three components: (a) the focused light exiting eye 2320 described above; (b) light originating from probe element 2302 reflected from various objects in the environment not shown, such as a viewer's face or nearby walls; and (c) light originating from sources other than probe element 2302, for example a lamp or the sun.

The signals output by light sensor group 2308 are processed by demodulation element 2342, which removes the components of the signals caused by unmodulated light originating from sources other than probe element 2302.

The outputs of demodulation element 2342 are then processed by comparison element 2344.

At this point the signals contain only components (a) and (b) described above. However, the component (b) resulting from non-image-forming reflections is nearly the same for all light sensors because the dimensions of the light sensors are small compared to the distance to nearby objects and because non-image-forming reflections quickly spread out to illuminate a given area evenly. The tendency of light from non-image-forming reflections to illuminate small areas evenly is illustrated in FIG. 25 where a small light source 2502 illuminates a larger surface 2504. The light reflected from surface 2504 is sampled at a group of closely spaced points 2506. It can be intuitively seen that the illumination at points 2506 from reflections off surface 2504 is relatively unvarying, as each point receives light from the whole of surface 2504. Accordingly, component (b) makes an approximately equal contribution to the signal output from each light sensor. Therefore the signal from light sensor 2312 is of greater magnitude than the signals from light sensors 2310 and 2314 as it corresponds to the brightest location in probe pattern 2350.

Comparison element 2344 subtracts the signal of light sensor 2310 from that of light sensor 2312 and then subtracts the signal of light sensor 2314 from that of light sensor 2312, forming first and second difference signals, respectively. The signs of the first and second difference signals are compared to third and fourth difference signals similarly computed for locations on the probe pattern corresponding to the locations of light sensors 2310, 2312, and 2314. The magnitudes of the first and second difference signals are nearly equal for reasons stated above and are determined by several factors including the amount of light output by probe element 2302 in the direction of eye 2320, the distance to eye 2320, the pupil diameter of eye 2320, and the gaze direction of eye 2320. Large magnitudes are indicative of any of the following: high light output from probe element 2302, short distance to eye 2320, large pupil diameter of eye 2320, and a gaze direction close to including probe pattern 2350. The difference signal magnitudes are compared to a minimum value and a maximum value. The minimum value may be used to prevent noise in the signals from producing false positives, or to set a maximum allowable distance from the probe pattern to eye 2320. The maximum value can be used to set a minimum allowable distance to eye 2320. Appropriate minimum and maximum values must be chosen uniquely for each application of the present invention and are best determined experimentally. One method to determine appropriate values is to measure signal magnitudes with a viewer present at various distances under various lighting conditions, but many appropriate methods exist and will be obvious to a practitioner skilled in the art.

When either or both difference signal magnitudes fail to satisfy the minimum or maximum value requirements a gaze is determined to not be present. When both difference signal magnitudes do satisfy the requirements, their signs are compared to the signs of the third and fourth difference signals. When the signs of the first and second difference signals match the signs of the third and fourth difference signals, respectively, a gaze is determined to be present and the average of the first and second difference signal magnitudes serves as a measure of the “gaze strength.” When either or both of the first and second difference signal signs differ from those of the third and fourth difference signals a gaze is determined not to be present.

Note that as illustrated in FIG. 23, beamsplitter 2306 transmits light from probe element 2302 and reflects light towards light sensor group 2308; however, configurations where light from probe element 2302 is reflected and light incident on light sensor group 2308 is transmitted are functionally equivalent.

As seen by a viewer, the angular separation of probe pattern 2350 and the point at which gaze tracking information is desired should be as small as possible and is preferably less than 45 degrees. As the angular separation increases, the amount of light reflected by eye 2320 to form an image decreases, and the image gets blurrier, making detection more difficult.

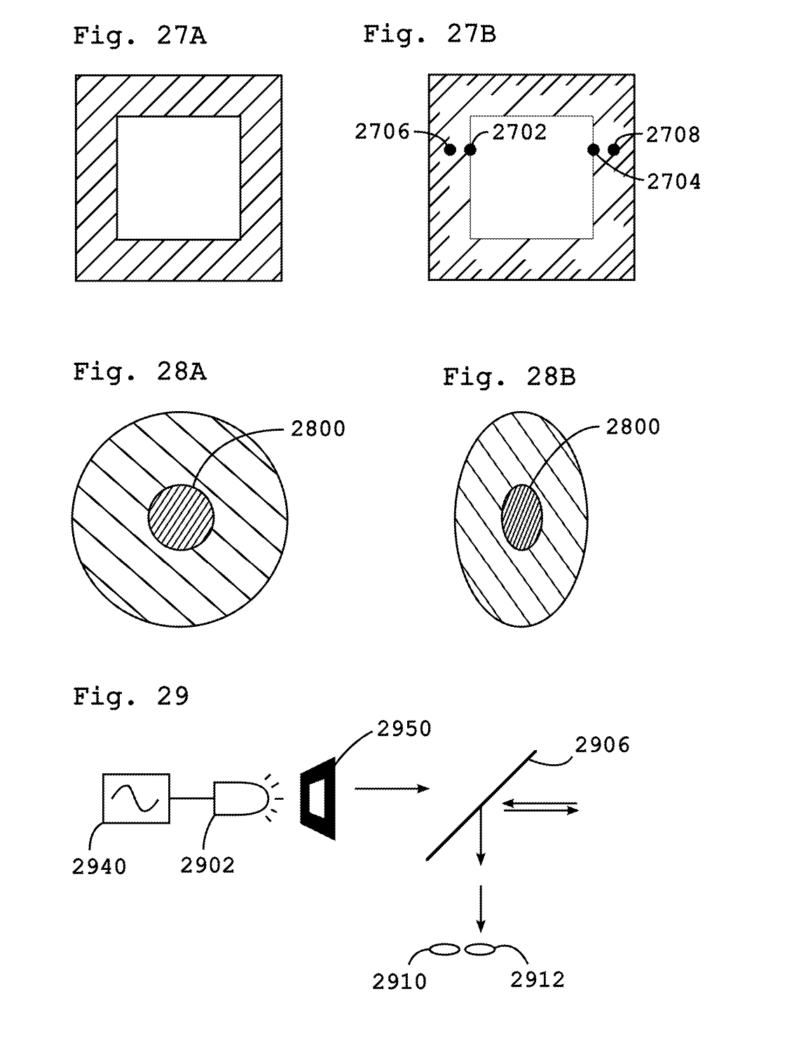

FIGS. 27A and 27B illustrate another principle relevant to the present invention. FIG. 27A shows a detailed view of probe pattern 2350. Until this point, image 2328 formed on retina 2326, and consequently the image projected by eye 2320, has been assumed to be merely a smaller version of probe pattern 2350. However, scattering effects, imperfections in cornea 2322 and lens 2324, and reflections within eye 2320 all conspire to distort or “blur” the image of probe pattern 2350 shown in FIG. 27A. One particularly import effect is sub-surface scattering. This effect is familiar to anyone who has pressed a flashlight to their hand and seen a red halo around the body of the flashlight. The halo occurs because light is not completely absorbed at the surface of the skin but is partially transmitted and scattered within the skin. Part of the scattered light re-emerges from the surface forming the red halo. A similar effect occurs in the tissues forming retina 2326. Sub-surface scattering and other effects including those mentioned above cause the image projected by eye 2320 to be blurred in a manner similar to that shown in FIG. 27B.

Another effect contributing to the blurred image of FIG. 27B is the fact that the focal lengths of lensed systems generally vary with the wavelength of light. When the light used to create the probe pattern is substantially different in wavelength from light on which the viewer is fixated, the image of the probe pattern formed at the viewer's retina will be slightly out of focus. The eye acts to project this out-of-focus image of the probe pattern as described above, and the image formed is further out of focus.

An alternative embodiment takes advantage of this effect, replacing probe element 2302 and light sensing element 2304 shown in FIG. 23 with the structure shown in FIG. 26. A light emitting element 2602 is surrounded by four light sensors 2610, 2612, 2620, and 2622. Light emitting element 2602 creates an image similar to that of FIG. 27A such that when a gaze is present an image similar to that of FIG. 27B is projected. Light sensors 2610 and 2612 correspond to points 2702 and 2704 in FIG. 27B, while light sensors 2620 and 2622 correspond to points 2706 and 2708 also in FIG. 27B. Points 2702 and 2704 are of an equal first intensity and points 2706 and 2708 are of an equal second intensity. The first intensity is greater than the second intensity. Light emitting element 2602 is modulated as described above for the preferred embodiment. Signals from the light sensors are processed in a manner similar to that described above for the preferred embodiment with first and second difference signals calculated for light sensor pairs 2610, 2620 and 2612, 2622. Gaze presence is determined by detecting the condition where the signals from light sensors 2610 and 2612 are greater than the signals from light sensors 2620 and 2622, respectively. Gaze strength is given by the average magnitude of the first and second difference signals.

Another embodiment operates in a similar manner to the embodiment of the last paragraph detecting a blurred reflection of a probe pattern. In this case, however, the reflection is caused to be blurred by deliberately locating either or both the probe pattern and light sensing element at optical path lengths from a viewer different from the optical path length from the viewer to a point where gaze information is desired. This might be accomplished, for example, by placing the embodiment of the previous paragraph slightly behind a measurement point where gaze information is desired, relative to the viewer. In this case the image of the probe pattern on the viewer's retina will be out of focus when the viewer focuses on the closer measurement point. The already out-of-focus image of the probe pattern will come to a focus at the measurement point and then diverge to be further out-of-focus upon reaching the more distant light sensing element.

Still other embodiments periodically record the demodulated signal level from each light sensor when a gaze is determined not to be present. The most recent such values are subtracted from the corresponding demodulated signals by the comparison element each time a comparison is made. The signal levels recorded when a gaze is not present are the result of non-image-forming reflections of the probe pattern. This technique can reduce or eliminate signal components due to reflections from nearby stationary objects, protective coverings over the device, a viewer's face, or other objects in the environment. The signal levels might be recorded when a viewer blinks for example, which may be detected as the brief absence of gaze.

In a further embodiment of the invention, the difference of light sensor signals to be compared is computed before reaching the comparison element, which then compares the differences to expected values. The difference of signals generally has a smaller dynamic range in varying ambient light conditions, so computing the difference earlier allows the use of less expensive hardware or lower circuit voltages where appropriate.

Another embodiment varies the probe pattern according to the distance of the viewer. As described above, the image of the probe pattern projected by the eye is slightly blurred, becoming more blurred as the image on the retina becomes smaller, an effect which occurs as a viewer becomes more distant from the probe pattern. The projected image may become so blurred as to be unrecognizable. To account for this situation, the probe pattern is periodically varied within a set of patterns appropriate for different viewer distances and the most appropriate, i.e., the pattern producing the highest gaze strength, is selected. The size of the selected probe pattern is related to and may be used to estimate the viewer distance from the probe pattern, information that is useful in many situations.

A further embodiment emits light forming the probe pattern only in certain directions where a viewer is either expected or known to be located. This saves power by only emitting light where needed, and at the same time reduces unwanted reflections from objects in the environment. The emitted direction may be scanned when a gaze is not detected in order to discover the direction of a viewer relative to the probe pattern. The direction of emission may then be continuously adjusted to track the viewer. The adjustment is performed by scanning the direction of emission in a small circle centered around the current emission direction which is then updated with the direction producing the strongest gaze detection signal. Note that this procedure may be used to track multiple viewers using a single device of the present invention by keeping a record of the directions of all known viewers and sequentially measuring the gaze of each viewer. Methods of scanning the emitted direction include movable mirrors, lenses, and diffractive optical elements, as well as many other methods familiar to those skilled in the art.

Still another embodiment places several of one of the above embodiments in a region where gaze is to be precisely tracked. In this embodiment the gaze is determined to be closest to the probe image with the highest “gaze strength.” As noted above, gaze strength depends on a number of factors, but the most relevant factor in this embodiment is gaze direction. FIG. 28A shows that, seen from the location of a probe image, a viewer's pupil 2800 appears circular when the viewer is looking at the probe image. FIG. 28B shows the same view when the gaze is averted; pupil 2800 appears elliptical. More light from the probe pattern can enter and exit pupil 2800 as shown in FIG. 28A than as shown in FIG. 28B, illustrating that the gaze strength is greatest at the tracked location closest to the gaze of the viewer.

Another embodiment places one or more of one of the above embodiments in a region where information concerning the number of viewers present is desired. This information is useful, for example, in the evaluation of advertising in a public place. In this embodiment the gaze strength at a measurement location or locations is recorded over time. The gaze strengths are then compared to experimentally determined values for known numbers of viewers to estimate the number of viewers present as a function of time.

The embodiments described above may be further appreciated in light of the following examples.

### EXAMPLE 1

A prototype of the system in FIG. 29 was constructed and tested. Commonly available infrared photodiodes BPW34F were used for light sensors 2910 and 2912. Commonly available LED TLN233 was used for a light source 2902, the emissive area of which forming an image 2950. LED TLN233 and photodiodes BPW34F are commonly available from distributors of electronic parts such as RS Components. Light source 2902 was pulsed for 1 ms at 100 Hz with approximately 250 mA of current. A beamsplitter 2906 was constructed from a half-mirrored acrylic sheet 2 mm in thickness. The signals from the photodiodes were pre-amplified by a factor of 10-100 million using TL084 JFET-input op-amps in a transimpedance amplifier configuration. The signals were bandpass-filtered to pass the 1 kHz pulses from light source 2902. The difference in signals from light sensors 2910 and 2912 was taken in several experiments. The differences were measured in millivolts using an oscilloscope. One experiment measured the system response to gaze in darkened conditions. The component due to image-forming reflections from a single viewer's retina at distances of 10-100 cm from the light source was found to be between 1 and 5 millivolts. A similar test in a room indirectly lit by sunlight on a fair day gave a detectable signal between 1 and 3 millivolts.

Another experiment was also conducted in darkened conditions with the eye approximately 20-40 cm from the light source. The point of focus was varied between approximately 10 cm from the eye to infinity along a line including the light source. A peak was found to be between 1 and 4 millivolts when the point of focus was at the light source, with smaller values when out-of-focus in both directions.

Still another experiment showed a falloff in measured gaze strength as the gaze was directed further and further from the probe target, with no detectable gaze when the gaze direction was averted more than 80 degrees.

The embodiments described above may be further appreciated in light of the following usage scenarios:

Usage Scenario 1

A mobile telephone left on a table receives a call and begins to emit an alert noise. One or more nearby persons look at the phone, a fact which is detected by a gaze detector of the present invention embedded in the phone. The phone ceases emitting noise after nearby persons look at the phone continuously for more than several seconds.

Usage Scenario 2

Personal computers are often configured with screen savers that activate after a given period of time, obscuring what was on the screen at the time activated. This behavior is often undesired, for example when reading text from the computer screen. A gaze detector may be embedded near the screen to detect visual attention by a user, disabling the screen saver when such visual attention is present.

Usage Scenario 3

Electrical or other devices including light switches, televisions, and music players may be controlled by movements of the eyes by providing one or more gaze detectors in communication with the device to be controlled. In order to prevent false activations a set of predefined eye movements may be detected and mapped to various functions. For example, two blinks in rapid succession detected as described above may turn the controlled device on or off. Visual attention directed to elements in a set of visual markers in a prescribed order may be mapped, for example, to changes in volume or selected channel.

Usage Scenario 4

A sleepy automobile driver begins to fall asleep while driving. A gaze tracker mounted in the driver's field of view detects the condition where the driver's eyes are closed for longer than several seconds and sounds an alarm, waking the driver.

Conclusion

Thus the coordinate input device of the present invention provides a highly economical device capable of discerning many different distributions of incident light, while the gaze tracking device of the present invention provides a highly economical and compact device.

Patents, patent applications, or publications mentioned in this specification are incorporated herein by reference to the same extent as if each individual document was specifically and individually indicated to be incorporated by reference.

While the above description contains many specificities, these should not be construed as limitations on the scope of the invention, but rather as exemplifications of preferred embodiments of the invention. Many other variations are possible.

What is claimed is:

1. a coordinate input device comprising:
a first medium capable of propagating light through total internal reflection;
a second medium capable of propagating light through total internal reflection arranged proximate to said first medium such that a force applied to said first medium directed towards said second medium causes light propagated by said first medium to be communicated to said second medium;
at least one photosensor producing an output signal or signals, said photosensor arranged to receive light propagated by said second medium; and
a processing means configured to receive said output signal or signals.

2. a gaze tracking device comprising:
a means of forming a probe image;
a means of measuring a distribution of light at a location proximal to said probe image, said distribution of light having originated from said probe image and having reflected from objects in an area surrounding said probe image; and
a processing means configured to compare said distribution of light to said probe image.
